(19) United States
(12) Patent Application Publication
(10) Pub. No.: US 2002/0072730 A1
(43) Pub. Date: Jun. 13, 2002

(54) Methods and apparatus for protecting the proximal end of a medical device

(76) Inventors: Scott A. McGill, Redwood City, CA (US); Jeffrey A. Nool, San Jose, CA (US); Mukund R. Patel, San Jose, CA (US); Arlene L. Errazo, Sunnyvale, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/976,679

(22) Filed: Oct. 12, 2001

Related U.S. Application Data
(60) Provisional application No. 60/239,665, filed on Oct. 12, 2000.

(30) Foreign Application Priority Data
Oct. 12, 2001 (WO) PCT US01/42690

(51) Int. Cl. A61M 25/00

(52) U.S. Cl. 604/525; 604/526

(57) ABSTRACT

Medical devices are provided with either a back end coil support or a sleeve support to prevent kinking of an inner wire used with the medical device. The medical device generally comprises an elongate tubular body having a lumen and an inner wire extending within the lumen. An expandable member is connected to the distal end of the tubular body. In one embodiment, a coil extends over the inner wire at the proximal end thereof. In another embodiment, the inner wire at its proximal end has at least one taper that increases the diameter of the inner wire to a size larger than the diameter of the lumen of the hypotube through which it extends. In another embodiment, a proximal hypotube is attached to the proximal end of the inner wire.

### CROSS-REFERENCE TO RELATED APPLICATION

This application claims the benefit of U.S. Provisional Application No. 60/239,665, filed Oct. 12, 2000, the entirety of which is hereby incorporated by reference.

### BACKGROUND OF THE INVENTION

1. Field of the Invention

This invention relates generally to the field of intravascular devices, and more particularly, relates to occlusive and other medical devices incorporating an expandable member used, for example, for emboli containment.

2. Description of the Related Art

Although attempts have been made to treat occlusions in the carotid arteries leading to the brain, such arteries have been very difficult to treat because of the possibility of dislodging plaque which can then enter various arterial vessels of the brain and cause permanent brain damage. Attempts to treat such occlusions with balloon angioplasty have been limited because of such dangers. In surgical treatments, such as endarterectomy, the carotid artery is clamped on either side of the treatment area, slit open and plaque is removed from the vessel in the slit area. Such surgical procedures, while being relatively safe from escape of emboli, nonetheless entail substantial risk.

In other procedures, such as in angioplasty and in the treatment of peripheral arteries and veins, there is the possibility that the delivery of the guide wires and catheters used in such procedures may dislodge plaque. When emboli or other particulates flow downstream to occlude blood flow in smaller vessels, they can cause serious damage, such as stroke. Thus, embolization and migration of micro-emboli downstream to an end organ is a major concern of cardiologists during catheterizations.

Various vascular devices have been proposed which would contain emboli produced as a result of intravascular procedures. However, the proper deployment of such devices remains problematic. For example, when a filter device is used, if a filter expands too far, damage to the vessel can result. Moreover, for a filter device using a pull wire to deploy the filter, kinking can result as the pull wire is advanced into an outer hypotube. Similarly, kinking can result in occlusion balloon devices and other balloon devices which use an inner wire moveable within an outer catheter body. Such inner wires may find use, for example, as a valve mechanism for sealing the lumen of the catheter.

Thus, there remains a need for new and improved apparatuses and methods which overcome these problems.

### SUMMARY OF THE INVENTION

The preferred embodiments of the present invention advantageously provide medical devices such as balloon catheters and filters for use in emboli containment and other types of procedures. The design of these devices preferably comprises at least two coaxially disposed elongate members, at least one of which is attached to the deployable expandable member. In a filter design, deployment of the occlusion device is achieved by the relative motion between the two elongate members, using relative axial translation, relative axial rotation, or both. The inner elongate member may be referred to as the pull wire, while the outer elongate member may be referred to as the hypotube. In a balloon design, the inner member or wire may extend out from the proximal end of the hypotube, and may serve as a valve for sealing the lumen of the hypotube, or may include a plunger which when advanced distally, inflates the balloon.

In a preferred embodiment, a coil is coaxially existent over the inner wire and bonded between the proximal end of the hypotube, which may also be referred to as the distal hypotube, and to a separate, proximal hypotube which extends over the rest of the inner wire proximally. In this embodiment the proximal hypotube is crimped to the inner wire and thus can be used to manipulate its relative position longitudinally to the distal hypotube. The coil serves both to add rigidity to the inner wire as well as to limit the range of relative motion between the hypotube and inner wire. This also ensures that the inner wire is not accidentally removed from the hypotube. For use in a filter device, this thereby prevents overexpansion of the filter device in the artery or vein.

In another preferred embodiment, the inner wire comprises at least two closely spaced tapers just proximal to the proximal end of the hypotube, allowing for a larger diameter of the inner wire beyond the proximal end of the hypotube. A thin-walled support tubing with a slightly larger inner diameter than the hypotube is attached to the proximal end of the hypotube extending proximally over a portion of the inner wire. This provides additional support to prevent kinking and/or bending of the inner wire at the point of insertion into the distal hypotube. This purpose is also served by the larger diameter of the pull wire due to the tapers.

Thus, in one aspect of the present invention, a medical device is provided comprising an elongate tubular body having a proximal end and a distal end and a lumen extending therethrough. An inner wire is provided within the lumen of the elongate tubular body having a proximal end extending proximal to the proximal end of the elongate tubular body and a distal end. The inner wire is moveable relative to the elongate tubular body. An expandable member is connected to the distal end of the elongate tubular body. A coil extends over the inner wire at a proximal end thereof, the coil being attached to the proximal end of the elongate tubular body. This coil preferably prevents kinking of the inner wire as it is moved relative to the elongate tubular body, and also prevents the inner wire from being removed from the elongate tubular body.

In another aspect, a medical device is provided comprising an elongate tubular body having a proximal end and a distal end and a lumen extending therethrough. A pull wire is provided within the lumen of the tubular body having a proximal end extending proximal to the proximal end of the tubular body and a distal end. An expandable occlusive device having a proximal end connected to the tubular body and a distal end connected to the pull wire is provided. Relative movement of the pull wire with respect to the tubular body causes the occlusive device to move from a nonexpanded configuration to an expanded configuration. The pull wire at its proximal end has at least one taper that increases the diameter of the pull wire to a size larger than the diameter of the lumen.

In another aspect of the present invention, a medical device is provided comprising an elongate tubular body having a proximal end and a distal end and a lumen extending therethrough. An inner wire within the lumen of the elongate tubular body has a proximal end extending proximal to the proximal end of the elongate tubular body and a distal end. The inner wire is moveable relative to the elongate tubular body. An expandable member is connected to the distal end of the elongate tubular body. A proximal hypotube is attached to the proximal end of the inner wire.

### BRIEF DESCRIPTION OF THE DRAWINGS

FIG. 1 is a partial sectional view of a shaft and filter subassembly deployed in a blood vessel, as well as a friction fit mechanism located proximal of the filter subassembly.

FIG. 2 is a side view of a strut hypotube of the filter subassembly.

FIG. 3 is a perspective view of the strut hypotube.

FIG. 4 is a sectional view of the strut hypotube, taken along the line 4-4 in FIG. 2.

FIG. 5 is a side view of a pull wire for use in the shaft and filter subassembly.

FIGS. 6 and 7 are partial cross-sectional views of a kink protection system for the pull wire, reflecting system conditions when the filter subassembly is in the contracted and expanded configurations, respectively.

FIGS. 8A-8C show an adapter for use with the shaft and filter subassembly of FIG. 1.

FIG. 9 illustrates another embodiment of an adapter for use with the shaft and filter subassembly of FIG. 1.

FIGS. 10 and 11 are partial cross-sectional views of another embodiment of a kink protection system for a pull wire similar to that of FIGS. 6 and 7, illustrating the contracted and expanded configurations, respectively.

FIGS. 12 and 13 are partial cross-sectional views of another embodiment of a kink protection system for a pull wire, illustrating the contracted and expanded configurations, respectively, wherein the pull wire is tapered.

FIGS. 14 and 15 are partial cross-sectional views of another embodiment of a kink protection system for a pull wire similar to that of FIGS. 12 and 13, illustrating the contracted and expanded configurations, respectively.

FIGS. 16 and 17 are partial cross-sectional views of another embodiment of a kink protection system for a pull wire, illustrating the contracted and expanded configurations, respectively.

FIGS. 18 and 19 are partial cross-sectional views of another embodiment of a kink protection system for a pull wire similar to that of FIGS. 16 AND 17, illustrating the contracted and expanded configurations, respectively.

FIGS. 20 and 21 are partial cross-sectional views of another embodiment of a kink protection system for a pull wire, illustrating the contracted and expanded configurations, respectively.

FIGS. 22 and 23 are partial cross-sectional views of another embodiment of a kink protection system for a pull wire, illustrating the contracted and expanded configurations, respectively.

FIG. 24 is a perspective view of an integrated inflation/deflation device, shown operably coupled to an illustrative inflation adapter and a balloon catheter deployed in a blood vessel.

FIG. 25A is a side view of a balloon catheter which can be used in accordance with one preferred embodiment of the present invention.

FIG. 25B is a longitudinal cross-sectional view of the distal end of the balloon catheter of FIG. 25A.

FIG. 25C is an enlarged cross-sectional view of the proximal end of the balloon of FIG. 25B.

FIG. 26 shows the inflation adapter of FIG. 24 having a low profile catheter valve and balloon catheter placed therewithin.

FIG. 27A is a partial cross-sectional view of a low profile catheter valve.

FIG. 27B is an enlarged view of the low profile catheter valve of FIG. 27A, showing the valve in an open position (and a closed position shown in phantom).

FIG. 28 is a side view of an illustrative single operator type aspiration catheter according to a preferred embodiment of the present invention.

FIG. 29 is a side cross-sectional view of a self-inflating balloon catheter according to one embodiment of the present invention.

### DETAILED DESCRIPTION OF THE PREFERRED EMBODIMENTS

The following description and examples illustrate preferred embodiments of the present invention in detail. Those of skill in the art will recognize that there are numerous variations and modifications of this invention that are encompassed within its scope. Accordingly, the description of preferred embodiments should not be deemed to limit the scope of the present invention.

I. Overview of a Filter Occlusion Device

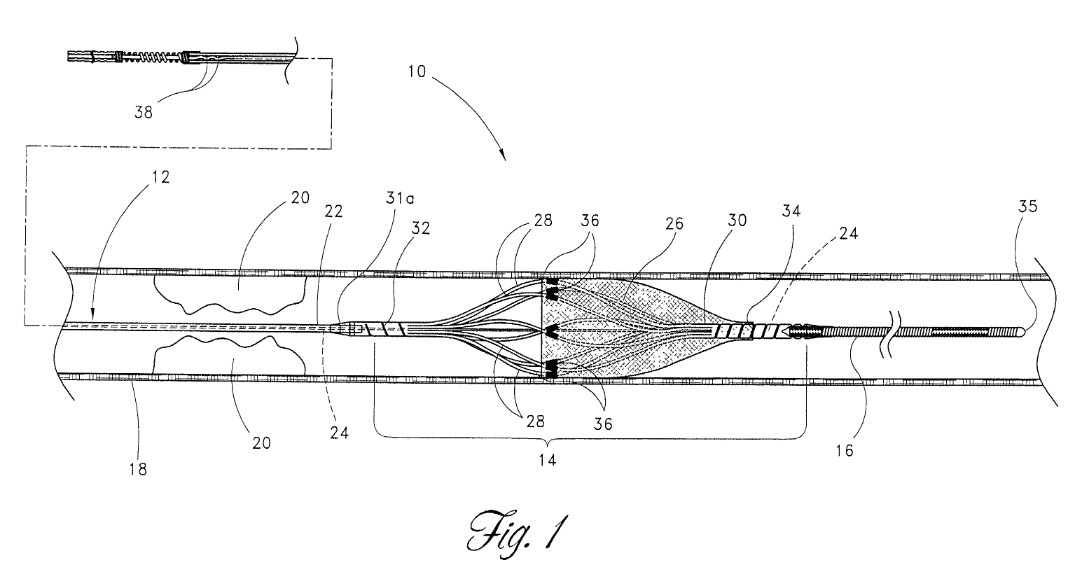

FIG. 1 illustrates a preferred embodiment of a filter device 10 comprising a shaft 12, a filter subassembly 14, and a guide tip 16. An adapter 118 (see FIGS. 8A-9) may be operably connected to the filter device to expand the filter. Further details of each of these components are described below.

In employing the device 10, the filter subassembly 14 is delivered on the shaft 12 to a location in a blood vessel 18 distal of an occlusion 20. Through the use of the adapter 118, the filter subassembly 14 is expanded to occlude the vessel distal of the occlusion. Various therapy and other catheters can be delivered and exchanged over the shaft 12 to perform treatment on the occlusion 18. Further details of this exchange are described in assignee's copending application entitled EXCHANGE METHOD FOR EMBOLI CONTAINMENT, Ser. No. 09/049,712, filed Mar. 27, 1998, the entirety of which is hereby incorporated by reference. Because the filter subassembly 14 remains expanded distal of the occlusion 18, any particles broken off by treating the occlusion 20 are trapped within the filter subassembly. These particles may then be removed by contracting the filter subassembly 14 so as to contain the particles and withdrawing the device 10 from the vessel. As an alternative or in addition to this method of particle removal, an aspiration catheter may be delivered over the shaft 12 and used to aspirate some or all of the particles from the filter subassembly 14.

A. Shaft

As shown in FIG. 1, the shaft 12 comprises an outer shaft member 22, and a pull wire 24 which extends through the lumen of the outer shaft member. The outer shaft member 22 may comprise a hypotube as is known in the art. Moreover, as described in assignee's copending application entitled STRUT DESIGN FOR AN OCCLUSION DEVICE, Ser. No. 09/505,546 filed Feb. 17, 2000, the entirety of which is hereby incorporated by reference, multiple hypotubes may be coaxially disposed over the pull wire 24. The shaft extends from a proximal end distally to the filter subassembly 14. The shaft may be constructed to any desired length, however, it is preferable for the shaft to be between about 120 and 300 cm in length.

The size of the outer member of the shaft 12 is suitable for insertion into the vasculature of a patient through an insertion site in the skin of the patient. It is preferable that the outer shaft member 22, the pull wire 24, and any other hypotube members are disposed coaxially such that each member is located within any larger diameter member and surrounds any smaller diameter member.

It is preferable that the largest diameter member of the shaft, for example outer member 22 in FIG. 1, has an exterior diameter of about 0.009 to 0.035 inches. It is more preferable that the largest diameter member of the shaft has an exterior diameter of about 0.012 to 0.035 inches, more preferably about 0.014 to 0.018 inches, and most preferably about 0.0142 inches. The wall thickness of the largest diameter hollow member of the shaft is preferably about 0.001 to 0.008 inches; i.e. the diameter of the lumen of the largest hollow member of the shaft is preferably from about 0.002 to 0.016 inches less than the outer diameter of the member. Any members located within the largest diameter member are preferably sized so as to fit within the inner lumen of the larger member.

As shown in FIG. 1, the outer member 22 of the shaft extends distally and is connected at its distal end to the filter subassembly 14. The pull wire 24 is the most centrally disposed of the shaft members. The pull wire 24 is preferably a solid, i.e. non-tubular member around which the outer member 22 is disposed. The pull wire 24 preferably extends inside the outer member 22, through the filter subassembly 14, and into the guide tip 16. Alternatively, the pull wire 24 may have two or more distinct segments, such as a proximal segment which extends to and terminates at the distal end of the strut hypotube 30 and a distal segment which extends from that point to the distal end of the guide tip 16.

The shaft members 22, 24 are preferably formed from a material which is sufficiently strong to support the shaft 12 itself as well as the filter subassembly 14 at the distal end under the tension, compression, and torsion experienced when inserting, operating, and removing the device from the vasculature of a patient. The material is preferably also sufficiently flexible and elastic that it does not develop permanent deformation while being threaded through the curved path necessary to reach the treatment site from the insertion point. In a preferred embodiment, the shaft 12 has a friction-reducing outer coating of TEFLON®.

In order to satisfy these requirements, it is preferable to use a metallic tube or wire to form the shaft members 22, 24, although a braided or non-braided polymer tube may also provide the desired characteristics. More preferably, a superelastic memory alloy such as straight-annealed nitinol is used for the outer shaft member 22; tempered stainless steel is one preferred material for the pull wire 24. Other suitable alloys for the shaft members include nitinol-stainless steel alloys, or nitinol alloyed with vanadium, cobalt, chromium, niobium, palladium, or copper in varying amounts. Additional details regarding materials used for the shaft members are disclosed in U.S. Pat. No. 6,068,623, the entirety of which is hereby incorporated by reference.

B. Filter Subassembly

Still referring to FIG. 1, the filter subassembly 14 extends from the distal end of the shaft 12. The filter subassembly 14 preferably comprises an expandable member which is either integrally formed or separately attached (as shown in FIG. 1) to the distal end of the shaft 12. The expandable member preferably includes an occlusive member or membrane 26 and provides support for this occlusive member.

As used herein, “occlusion” or “sealing”, and the like, refer to blockage of fluid flow in a vascular segment, either completely or partially. In some cases, a complete blockage of the blood vessel may not be achievable or even desirable, for instance, when blood flow must be maintained continuously to the region downstream of the occlusive device. In these cases, perfusive flow through the occluded region is desirable and a partial blockage is used. For example, a partial blockage may be produced using an occlusive member whose cross-sectional dimension does not span the entire blood vessel. Alternatively, a partial blockage may be produced using an occlusive member whose cross-sectional dimension does substantially span the entire blood vessel, but which contains openings or other means for flow to move through the occlusive member perfusively. In other cases, a partial blockage may not be achievable or desirable, and an occlusive member which substantially spans the cross section of the blood vessel without allowing perfusion is used. Each of these described structures makes use of “occlusion,” as defined herein.

In the embodiment shown in FIG. 1, the expandable member comprises struts 28 which are formed in a strut hypotube 30. The strut hypotube 30 extends from the distal end of the outer shaft member 22 to the proximal end of the guide tip 16. At its proximal end the strut hypotube 30 is soldered, crimped, and/or bonded, or otherwise affixed to the distal end of the outer shaft member 22. In a preferred embodiment, a proximal taper 31a, preferably formed from a flexible UV-cured adhesive, facilitates the connection of the strut hypotube 30 to the shaft 12. At its distal end the strut hypotube 30 is crimped over a solder junction between the pull wire 24 and the proximal end of the guide tip 16. A distal taper 31b, also preferably formed from a flexible UV-cured adhesive, may be employed as well in attaching the strut hypotube 30 to the guide tip 16. With the strut hypotube, pull wire and guide tip joined in this manner, a proximal movement of the pull wire with respect to the outer shaft member 22 causes a corresponding proximal movement of the distal end of the strut hypotube, thus compressing the strut hypotube and urging the struts toward the expanded position.

The strut hypotube 30 is preferably formed from nitinol, but may alternatively be formed from nitinol-stainless steel alloys, or nitinol alloyed with vanadium, cobalt, chromium, niobium, palladium, or copper in varying amounts. The strut hypotube preferably has an outside diameter of about 0.021 inches and an inside diameter of about 0.014 inches.

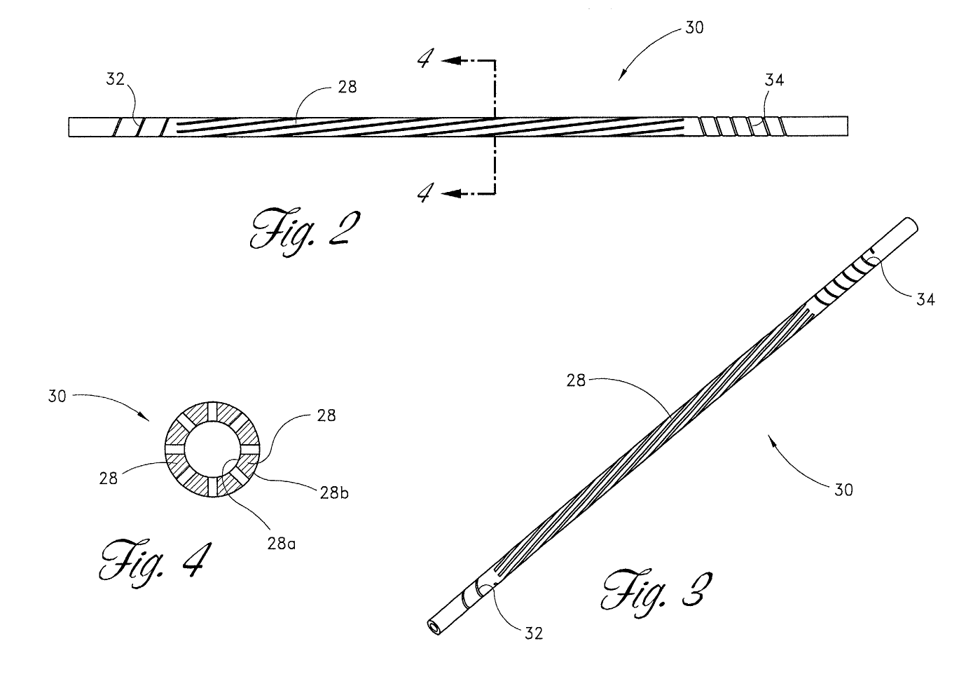

As best seen in FIGS. 2 and 3, the individual struts 28 are preferably cut from, and thus integral to, the strut hypotube 30. The struts 28 may advantageously be formed by subjecting the strut hypotube 30 to a laser-cutting process. Although the number of struts 28 may vary, there are preferably between 4 and 10 (most preferably 8) struts. The struts 28 should be equally radially spaced about the longitudinal centerline of the strut hypotube 30.

It is preferred that the struts 28 have a helical configuration, with each strut making approximately 1.0 revolution, at a substantially constant pitch, about the longitudinal centerline of the strut hypotube 30 as it extends from its proximal to its distal end. Alternative preferred embodiments have straight slits which provide for non-spiral struts when deployed into the expanded configuration. The preferred helical configuration improves the apposition of the struts against the vessel wall when the filter subassembly is in the expanded configuration. The struts 28 may advantageously have a constant clockwise pitch of about 0.650 inches and therefore the portion of the hypotube into which the struts are cut is about 0.650 inches in length. It is contemplated that the filter subassembly should reach a preferred maximum diameter of about 7.5 mm when expanded. As used herein, “strut” refers to any mechanical structure which extends from another structure or which is used to support a membrane or other structure of the occlusion device. Specifically, as discussed herein, the struts of the occlusion device are those portions of the device which extend from the shaft in order to adjust the profile of the device as discussed below, and which may be used to support the membrane.

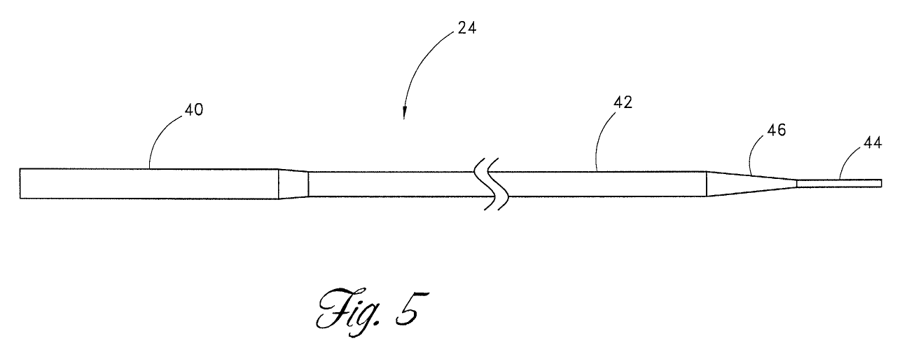

FIG. 4 depicts a cross-section of the strut hypotube 30, taken along the line 4-4 as shown in FIG. 2. The preferred laser-cutting process creates a gap of about 0.0018 inches in width between each pair of struts 28. Each strut 28 thus has a preferred cross-section that comprises an angular section of an annulus, with a smaller-radius inner surface 28a and a broader, larger-radius outer surface 28b. By virtue of their increase in size near the outer surface 28b, the struts 28 are stronger than a comparable set of struts that have a simple rectangular cross-section and are sized to fit within the same inner diameter-outer diameter “envelope.”

With further reference to FIGS. 2 and 3, the strut hypotube 30 may preferably incorporate a proximal cut 32 and/or a distal cut 34, to improve the flexibility of the hypotube. Each of the cuts 32, 34 is helical, with the proximal cut 32 having a preferred substantially constant pitch of about 0.030 inches and the distal cut 34 having a preferred substantially constant pitch of about 0.020 inches. The proximal cut 32 and distal cut 34 preferably extend along about 0.075 inches and 0.125 inches, respectively, of the hypotube 30 (as measured along its longitudinal axis), and each has a preferred cut width of about 0.0018 inches. Preferably, an uncut “gap” of about 0.015 inches exists on the strut hypotube 30 between the proximal cut 32 and the proximal end of the struts 28, and between the distal cut 34 and the distal end of the struts. As shown in FIG. 1, when the strut hypotube 30 is attached to the shaft 12 and the guide tip 16, it is advantageous that no part of the cuts 32, 34 overlie any portion of the shaft or guide tip, so as not to impede the flexibility enhancement that is provided to the strut hypotube by the cuts.

In a preferred embodiment, one or more marker bands 36 (see FIG. 1) are attached to a corresponding number of the struts, and are advantageously located at or near the midpoint of each strut, so as to align the marker bands with the widest portion of the filter subassembly 14 when it is in the expanded configuration. The marker bands may thus be aligned in a plane extending substantially orthogonal to the longitudinal axis of the shaft 12. Alternatively, the marker bands 36 may be staggered, i.e. attached in varying locations along the length of the struts 28, in order to reduce the profile of the filter subassembly when it is in the collapsed configuration. The marker bands are advantageously configured to wrap around only three sides of each strut, leaving the outer surface 28b (see FIG. 4) exposed, in order to reduce the profile of the filter subassembly when it is in the expanded configuration. A proximal marker band (not shown) may be incorporated in a location proximal of the struts 28 to mark a point on the device beyond which a catheter positioned on the shaft 12 should not be advanced, thus preventing inadvertent collapse of, or damage to, the struts 28. A preferred location for the proximal marker band is at the junction of the shaft 12 and the strut hypotube 30, underlying the proximal taper 31a. Additional details not necessary to mention here may be found in U.S. Pat. No. 6,228,072, the entirety of which is hereby incorporated by reference.

The marker bands 36 are formed from a material having increased radiopacity in comparison to the rest of the filter subassembly, such as platinum, gold, or alloys thereof. In a preferred embodiment, the marker bands comprise an alloy of 80% platinum and 20% iridium. Additional details not necessary to mention here may be found in assignee's copending application entitled VASCULAR FILTERS WITH RADIOPAQUE MARKINGS, Ser. No. 09/747,175, filed Dec. 22, 2000, the entirety of which is hereby incorporated by reference.

As shown in FIG. 1, the pull wire 24 extends past the distal end of the outer shaft member 22, beyond the strut hypotube 30, and terminates in a solder joint 35 at the distal end of the distal tip 16. The tip 16 distal to the struts 28 preferably includes a radiopaque coil material, most preferably platinum, extending between the distal end of the strut hypotube and the solder joint 35 to aid the practitioner in positioning the expandable member 14 within the vessel 18.

The membrane 26 is preferably attached at its proximal end to the struts 28, at or proximal of the struts' widest extent when in the expanded configuration. It is also preferred that the membrane 26 is attached at its distal end to the strut hypotube at or adjacent the distal cut 34. Between these proximal and distal points of attachment, the membrane tapers gradually to a smaller diameter but preferably tapers less sharply than the distal portion of the struts 28, so as to remain free from the struts, in a relatively loose or “baggy” state. When the expandable member is deployed, this “baggy” membrane creates a rather deep pocket for catching emboli as blood flows through the membrane 26, and for containing the emboli when the expandable member is collapsed and withdrawn from the vessel 18.

Alternatively, the membrane 26 may be attached to the struts 28 at one or more points, or in a continuous attachment, between the proximal and distal ends of the membrane. Many other arrangements are possible for the structure and attachment of the membrane 26. Reference may be made to assignee's copending patent applications Ser. No. 09/505,554, entitled MEMBRANES FOR OCCLUSION DEVICE AND METHODS AND APPARATUS FOR REDUCING CLOGGING, filed Feb. 17,2000, and Ser. No. 09/788,885, filed entitled MEMBRANES FOR OCCLUSION DEVICE AND METHODS AND APPARATUS FOR REDUCING CLOGGING, filed Feb. 20, 2001, the entirety of each of which is hereby incorporated by reference. As used herein, “filter” and like terms mean any system which is capable of separating something out of a portion of the blood flow within the vascular segment, whether or not there is perfusion through the “filter”. “Filtering” and similar terms refer to the act of separating anything out of a portion of the blood flow.

The membrane 26 has a number of pores (not shown) of a suitable size to trap emboli while permitting blood to flow through, and are thus about 20-100 microns in size. Suitable nonelastomeric materials for the membrane 26 include polyurethane, polyethylene, polyethylene terephthalate (PET), expanded polytetrafluoroethylene (PTFE), and polyether-based polyamides sold under the trade name PEBAX by Elf Atochem. One suitable elastomeric material is a block copolymer of styrene-ethylene-butylene-styrene (SEBS), available under the trade name C-FLEX, sold by Consolidated Polymer Technologies. The membrane may also be made from latex or silicone. The membrane may alternatively comprise a polymer mesh of polyurethane, nylon, polyester, or polyethylene, with pores approximately 30-50 microns in diameter. Yet another alternative is a braid of polyester or nitinol. To prevent formation of blood clots on the occlusive member, it may be coated with heparin or other known antithrombogenic agents such as hirudin or pirudin.

Most preferably, the membrane 26 is formed from polyurethane and has pores of about 100 microns in size, or a combination of pore sizes within the ranges detailed above. The pores are preferably spaced apart on the membrane with about 0.006 to 0.012 inches between the centers of adjacent pores, more preferably about 0.010 inches. It is also preferred that the proximal portion of the membrane lack pores, to facilitate bonding the membrane to the struts 28 over the marker bands 36. Likewise, the distal portion of the membrane may also be nonporous, providing easier attachment to the strut hypotube 30.

Further details not necessary to repeat here are disclosed in assignee's copending application entitled OCCLUSION OF A VESSEL, Ser. No. 09/374,741, filed Aug. 13, 1999, the entirety of which is hereby incorporated by reference.

C. Pull Wire

The outer shaft member 22 surrounds the pull wire 24 and is connected to the strut hypotube 30 at its proximal end (see FIG. 1). The pull wire 24 is advantageously attached to distal end of the strut hypotube 30, so that when the pull wire 24 is retracted relative to the outer shaft member 22, the struts 28 are urged to expand in a radial direction. The relative position of the outer shaft member 22 and the pull wire 24 is varied until the vessel 18 is occluded. The struts 28 bow outwards toward the wall of the vessel 18, so that the filter subassembly 14 seals the vessel 18 (i.e., in its deployed position, the expandable member prevents emboli from moving downstream). The radial expansion of the struts 28 may also be facilitated by advantageously imparting an initial curvature to the struts 28 through heat setting. The pull wire 24 may advantageously extend within the distal guide tip 16 beyond the distal end of the strut hypotube 30 and terminate in the solder joint 35 at the distal end of the guide tip.

After the filter subassembly 14 is deployed, the struts 28 tend towards their collapsed, undeployed position in the absence of a restraining force (unless the filter subassembly 14 is self-expanding, in which case the filter subassembly has a tendency to remain in the deployed position). To prevent the struts from returning to their undeployed position, the pull wire 24 has one or more bends 38 formed therein for contacting the inner wall of the outer shaft member 22, thereby providing frictional forces which keep the filter subassembly 14 in its expanded, deployed position, as shown in FIG. 1. Specifically, the frictional force between the pull wire 24 and the outer shaft member 22 is sufficient to offset or compensate for the spring force provided by the struts 28 and/or the membrane 26, which would otherwise urge the struts towards their relaxed position. About 0.5-1 pound of pulling force may be required to expand the struts 28. Thus, the bends 38 of the pull wire 24 engage the outer shaft member 22 to form a compact device for restraining the pull wire from unwanted longitudinal motion. The bends 38 of the pull wire 24 may be formed, for example, by coining or by forming a spring in the pull wire. The bends 38 thus act as a locking member which inhibits movement of the pull wire 24, and the pull wire 24 and the outer shaft member 22 are frictionally secured together.

The pull wire features of the embodiment of FIG. 1 can also be used if the filter subassembly 14 is shape set so that it tends toward an expanded, deployed position in the absence of any applied forces, i.e. if the expandable member is self-deploying. In the case where an embodiment such as that shown in FIG. 1 is constructed using a self-deploying filter subassembly 14, the pull wire 24 effectively acts as a push-wire which holds the filter subassembly in the collapsed configuration. This push-wire is held in place by the frictional engagement between the bends 38 of the pull wire and the outer shaft member 22.

When using such a device as shown in FIG. 1 with an expandable member which is self-deploying, the filter subassembly 14 is inserted into the vessel 18 of the patient in its low profile position, with frictional forces between the pull wire 24 and the outer shaft member 22 holding the pull wire 24 in the distal direction, which prevents the filter subassembly from expanding. The filter subassembly 14 is then deployed by urging the pull wire 24 in the proximal, axial direction (retracting the pull wire) with sufficient force to overcome the frictional forces between the pull wire 24 and the outer shaft member 22, thereby moving the locking member 38 out of its locked position. In effect, by moving the pull wire proximally in this way, the “pushing” effect of the pull wire is eliminated, and the expandable member will deploy into the expanded configuration.

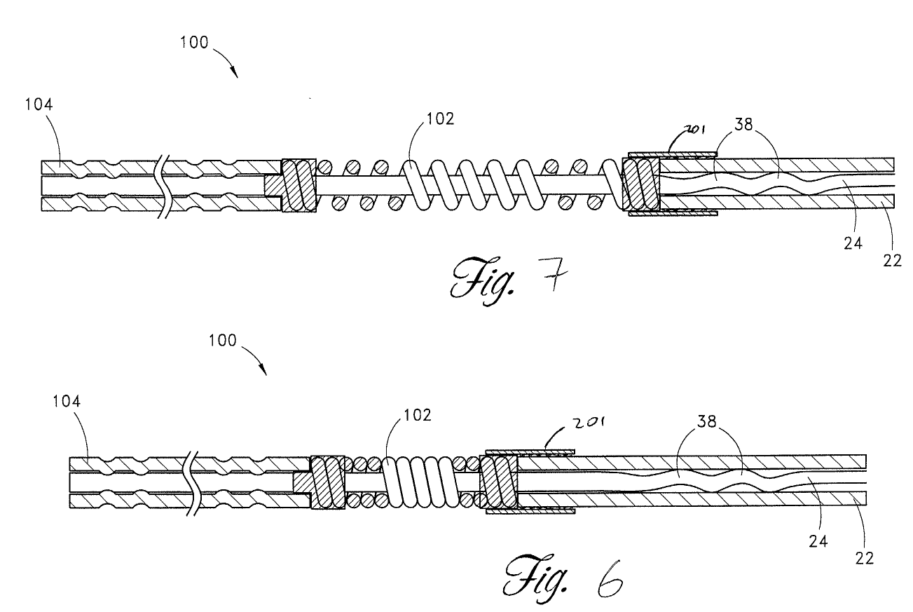

FIG. 5 shows one preferred embodiment of the pull wire 24. A preferred pull wire 24 comprises a tempered stainless-steel wire with an anti-friction coating of TEFLON®. This pull wire 24 has a tapered configuration, with a proximal section 40 having a diameter of about 0.0086 inches; advantageously, this larger-diameter proximal section of the pull wire includes the bends 38 described above. Distal of this section the pull wire tapers to a medial section 42 having a diameter of about 0.007 inches. The pull wire shown has a diameter of about 0.0025 inches at its most distal section 44; this diameter advantageously prevails over the most distal 3 cm of the pull wire. A tapered transition 46 of about 3 cm in length is interposed between the medial section and the distal section. The pull wire of FIG. 5 has an overall length of about 212.0 cm; the proximal section (having the diameter of about 0.0086 inches) is about 17.0 cm in length. The medial section is thus about 189.0 cm in length.

D. Pull Wire Kink Protection

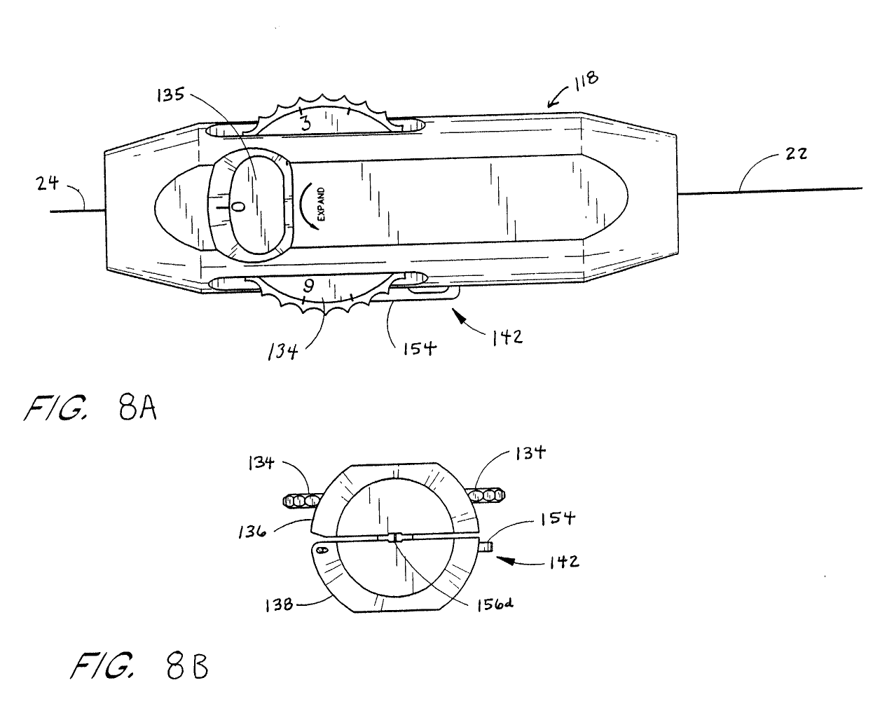

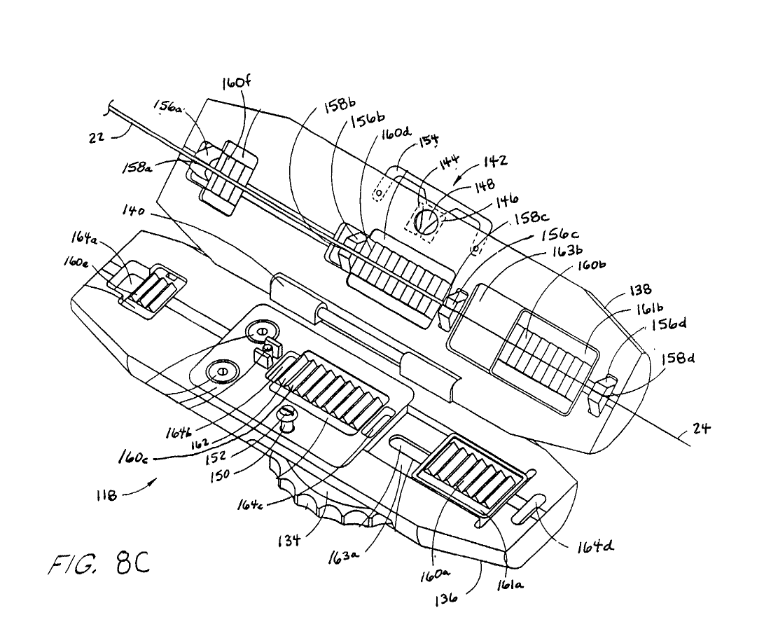

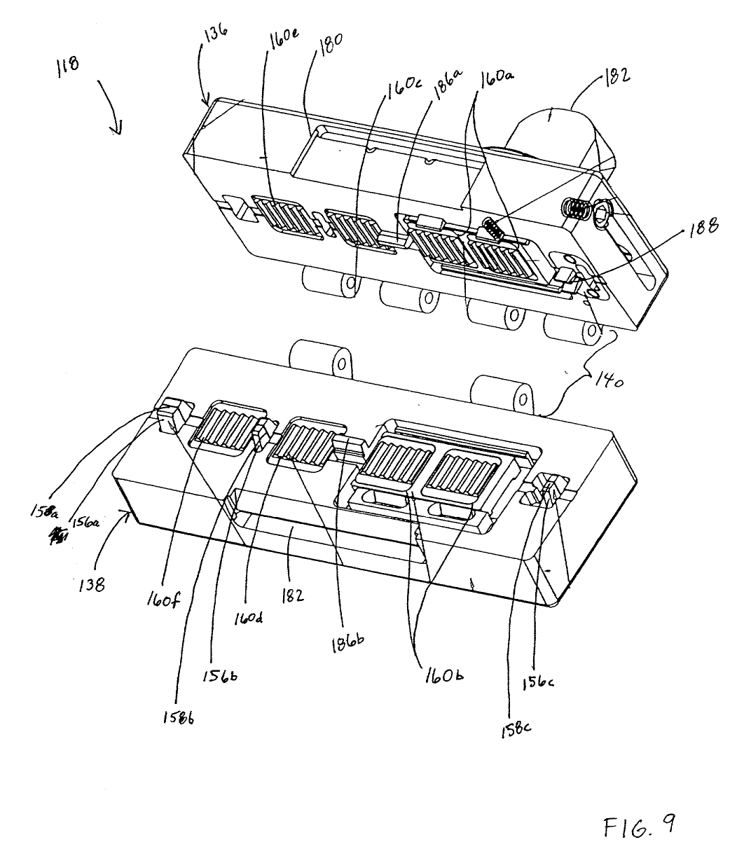

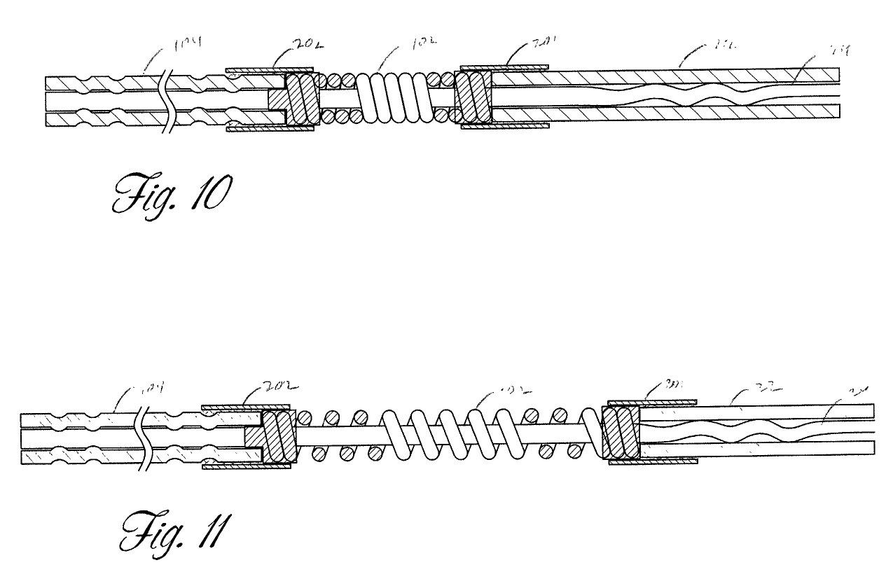

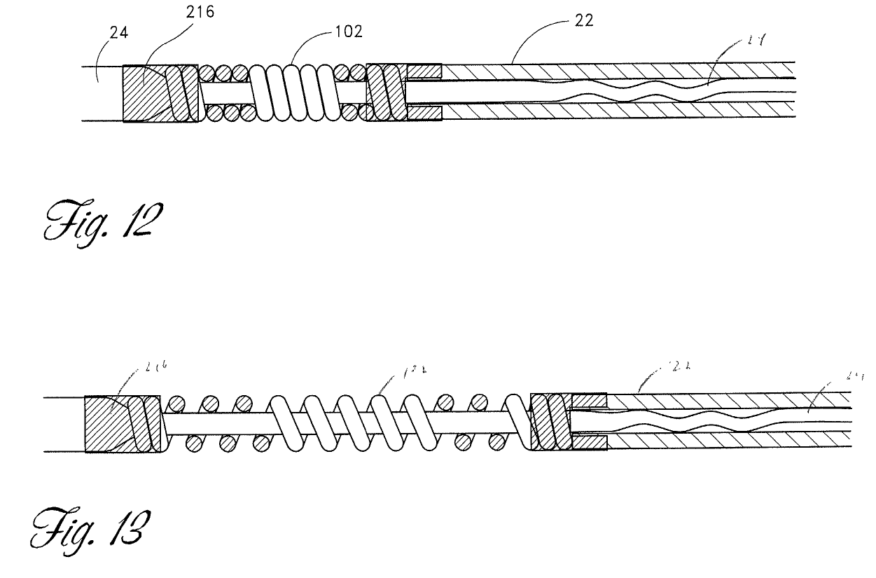

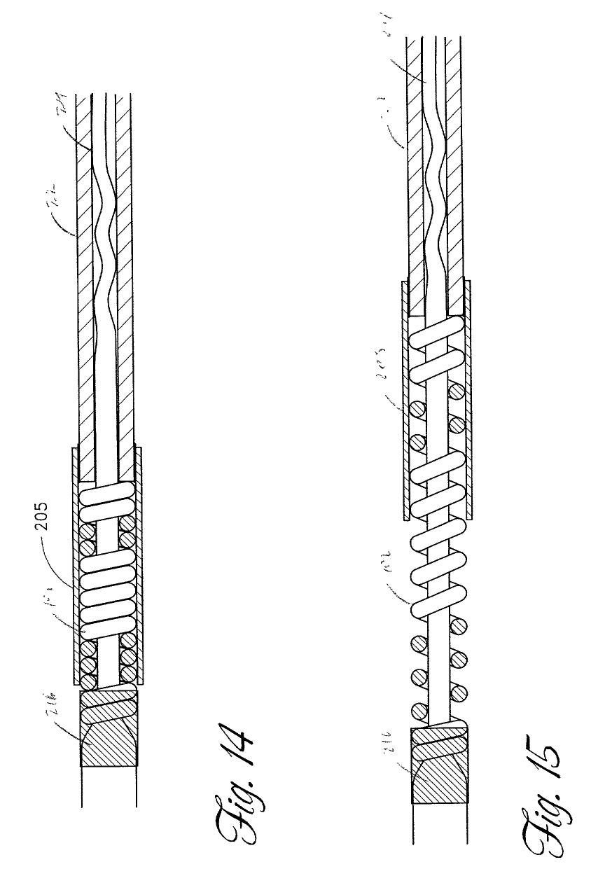

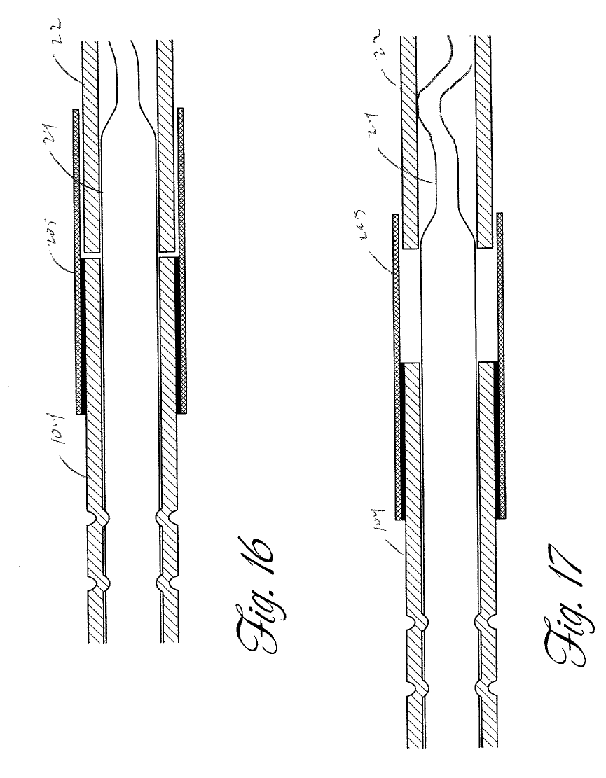

FIGS. 6 and 7 depict a kink protection system 100 that may preferably be used to prevent the proximal portion of the pull wire 24 from kinking when it is pushed distally against the frictional resistance of the bends 38 and, where the filter subassembly 14 is of the self-expanding type, against the spring force of the struts 28. The system 100 comprises a pre-expanded coil 102 and a proximal hypotube 104. The coil 102 is connected to the proximal tip of the outer shaft member 22 by soldering or other conventional methods and surrounds that portion of the pull wire 24 which is immediately proximal of the outer shaft member. The proximal hypotube 104 is crimped to the pull wire 24 and is attached to the proximal end of the coil 102 by soldering or other conventional methods.

FIG. 6 shows the system 100 when the filter subassembly is in its contracted configuration, and the coil 102 is compressed. FIG. 7 shows the system 100 when the filter subassembly is in the expanded configuration. The pull wire 24 has been pulled proximally from the outer shaft member 22 and the coil 102 is in its relaxed state. When the pull wire 24 is pushed back distally into the outer shaft member 22 (see FIG. 6), the coil 102 augments the column strength of the pull wire 24 by presenting a coaxial, larger-diameter column for absorbing the compressive force that is applied to the coil-pull wire assembly. Off-axis loads are thus less likely to bend or kink the pull wire 24 as it is pushed into the outer shaft member 22.

E. Adapter

The pull wire 24, shown in FIG. 1, is manipulated through the use of an adapter or manifold 118 (see FIGS. 8A-9). The adapter enables the technician to control the relative positioning of the pull wire 24 and the outer shaft member 22 in a simple manner. Although FIGS. 8A-9 illustrate the adapter as manipulating the pull wire 24, it will be appreciated that in embodiments wherein a proximal hypotube is provided over the pull wire 24, the adapter manipulates this proximal hypotube.

After delivery of the device to the desired location within the vasculature of the patient, the adapter 118 is attached and the pull wire 24 is manipulated through the use of the adapter 118 so as to deploy the filter subassembly 14 of the device. At this point, the adapter may be removed from the device so that therapy may be performed.

One type of adapter 118 used in accordance with preferred embodiments of the filter device is shown in FIGS. 8A-8C. Without regard to whether the expandable member is of the shape set variety (self-expanding) or is undeployed when relaxed, the degree to which the expandable member is deployed can be monitored by noting the longitudinal position of the pull wire 24. This allows the user to carefully control the extent to which the expandable member is deployed. A thumb wheel 134 is used to control the position of the pull wire 24 relative to the outer shaft member 22, thereby controlling the extent to which the filter subassembly 14 of FIG. 1 is expanded. As illustrated by the view of FIGS. 8B-8C, the adapter 118 includes two halves 136, 138 preferably formed of medical grade polycarbonate or the like.

The two halves 136, 138 are attached by at least one hinge 140, so that the halves are joined in a clam shell manner. A latch 142 secures the two halves 136, 138 while the adapter 118 is in use. The latch includes a pair of flexible, resilient latching members 144, 146 which are mounted within the half 138. A space 148 between the two latching members 144, 146 receives a locking pin 150 which has a beveled head 152. The head 152 passes through the space 168 and past the latching members 144, 146. The latching members 144, 146 prevent the locking pin 180 from backing out past the latching members which would open up the adapter 118. To open the halves 136, 138, the latching members 144, 146 are separated slightly by depressing a flexure member 154, which pries apart the latching members slightly, thereby freeing the locking pin 150.

The outer shaft member 22 may be held in place by a groove (not shown) having a width selected to accept the outer shaft member 22. Alternatively, as shown in FIG. 8C, the outer shaft member 22 and the pull wire 24 may be held by clips 156a, 156b, 156c, 156d having respective slots 158a, 158b, 158c, 158d therein for receiving the outer shaft member and the pull wire. In particular, the outer shaft member 22 and the pull wire 24 may advantageously be configured so that the outer shaft member rests within clips 156a, 156b, 156c, with the pull wire extending between the clip 156c and the clip 156d and extending proximal to the clip 156d. With this arrangement, and when the adapter 118 is in the closed position, the pull wire 24 may be engaged and moved by a first pair of contact members such as oppositely facing pads 160a, 160b, while the outer shaft member 22 is held stationary by one or more other pairs of oppositely facing pads 160c, 160d and 160e, 160f. Alternatively, the device may be designed so that the outer shaft member 22 is moved while the pull wire 24 remains stationary. The pads 160a-f may advantageously include a plurality of ridges 162 for securely contacting the pull wire 24. The clips 156a, 156b, 156c, 156d fit within respective cavities 164a, 164b, 164c, 164d in the adapter half 136 when the two halves 136, 138 are closed.

To aid the user in properly aligning the outer shaft member 22 and the pull wire 24 within the adapter 118, a mark may be placed on the outer shaft member 22. For example, an alignment mark on the outer shaft member 22 may indicate that point on the outer shaft member 22 which must be placed within the slot 158a so that the outer shaft member extends within the adapter 118 up to but not proximally beyond the clip 156c, with the pull wire 24 being exposed proximal to the clip 156c. This configuration permits the pads 160a, 160b to retract (or advance) the pull wire 24 into (or out of) the vessel while the outer shaft member 22 is held securely within the pads 160c, 160d and 160e, 160f.

When the pull wire 24 is not being advanced or retracted through the outer shaft member 22 by the pads 160a, 160b, relative movement of the pull wire and the outer shaft member is advantageously prevented by frictional contact between the bends 38 of the pull wire 24 and an inner surface of the outer shaft member 22 (see FIG. 1). This permits the introduction of a therapy catheter (not shown) such as an angioplasty or stent catheter, or the exchange of a plurality of catheters, after the adapter 118 is decoupled and removed from the outer shaft member 22 and the pull wire 24. For example, once the filter subassembly 14 is deployed, an angioplasty or stent catheter may be introduced over the outer shaft member 22 and the pull wire 24. After therapy is performed, an aspiration (and/or irrigation catheter) may be introduced over the outer shaft member 22/pull wire 24 to aspirate (and/or irrigate) away emboli entrained in the filter subassembly 14 which were produced as a result of the therapy procedure. The adapter 118 may then be recoupled to the outer shaft member 22 and the pull wire 24, followed by deactivation (retraction) of the filter subassembly. The filter subassembly 14, the pull wire 24, and the outer shaft member 22 may then be removed from the vessel.

When the adapter 118 is in the closed position, the pads 160c, 160d, 160e, 160f surround and contact the outer shaft member 22 to prevent its motion. The pads 160a, 160b, on the other hand, are mounted in respective holders 161a, 161b which are slidable within respective recessed portions 163a, 163b of the adapter 118, so that when the pads 160a, 160b, surround and contact the pull wire 24, the pull wire may be retracted or advanced. Specifically, the holder 161a (housing the pad 160a) is mechanically coupled to and controlled by the wheel 134, as discussed in more detail below. When the adapter 118 is closed, the pads 160a and 160b are compressed together and squeeze the pull wire 24 between them. As the user rotates the wheel 134, the pad 160a is moved in the longitudinal direction, and the pad 160b and the pull wire 24 are moved along with it. Thus, by rotating the wheel 134, the user may control the longitudinal position of the pull wire 24 with respect to the outer shaft member 22, and thereby control the extent to which the expandable member is radially deployed. The pads 160a-f may be formed from C-Flex or Pebax and are preferably about 0.5-1.0″ long, 0.25-0.5″ wide, and 0.125-0.25″ thick.

The wheel 134 imparts motion via a cam mechanism (not shown) to the pad 160a which moves the pull wire 24 incrementally. The wheel 134 may advantageously move the pull wire 24, for example, between 3 mm and 20 mm as indicated by a dial 135 on the face of the wheel (see FIG. 8A), thereby controlling the extent to which the expandable member is expanded by controlling the position of the pull wire. The dial 135 acts as a gauge of the relative longitudinal position of the pull wire 24 within the vessel, and thus as a gauge of the extent to which the expandable member has been expanded.

Another embodiment of the adapter 118 is shown in FIG. 9. This embodiment has the same basic configuration as that shown in FIGS. 8A-8C, i.e., a clamshell with two halves 136, 138 rotatably connected by at least one hinge 140. A resilient locking clip (not shown) may be mounted in a recess 180 formed in the upper half 136 and extend downward therefrom. Upon closure of the adapter 118 an inwardly-extending tongue formed on the locking clip snaps into a groove 182 formed in the lower half 138. The locking clip holds the adapter 118 firmly closed by virtue of an interference fit between the tongue and the groove 182.

In place of the thumb wheel 134 shown in FIGS. 8A-8C, this embodiment of the adapter 118 incorporates a knob 184 that is rotated by the user to move the pads 160a, 160b and advance/retract the pull wire 24. Like the thumb wheel 134, the knob 184 may incorporate appropriate markings (not shown) to indicate the extent to which the filter has been expanded or retracted by the action of the adapter 118.

Like the adapter shown in FIGS. 8A-8C, the adapter 118 of FIG. 9 includes pads 160c, 160d, 160e, 160f that grip the outer shaft member and hold it stationary while the pull wire is advanced or retracted within it. Clips 156a, 156b, 156c having respective slots 158a, 158b, 158c receive the outer shaft member and/or pull wire and maintain it in a straight configuration for the filter deployment/retraction process. Upper and lower channel halves 186a, 186b coact to create, upon closure of the adapter 118, a channel that receives and grips the outer shaft member and the pull wire, preferably immediately adjacent the pads 160a, 160b.

A pin member 188 is positioned on the upper half 136 so that the pin 188 is depressed by the pull wire when the adapter 118 is closed with the outer shaft member and pull wire positioned therein. The pin member is mechanically coupled to an interrupt mechanism (not shown) that prevents rotation of the knob 182 unless the adapter 118 is closed with the pull wire, etc. in position (and the pin member 188 depressed by contact with the pull wire).

Additional details not necessary to repeat here are disclosed in assignee's copending application entitled OCCLUSION OF A VESSEL AND ADAPTER THEREFOR, application Ser. No. 09/505,911, filed Feb. 17, 2000, the entirety of which is hereby incorporated by reference.

F. Strut Design

With further reference to FIG. 1, the filter device includes a filter subassembly 14 which is located along the shaft 12 near the distal end, and proximal of the guide tip 16. In one embodiment the filter subassembly may be integrally formed with the outer member 22 of the shaft 12. The filter subassembly 14 comprises a number of struts 28 and an occlusive member or membrane 26. The struts support the membrane, and provide for at least two configurations of the device, a collapsed configuration and an expanded configuration. The expanded configuration is shown.

The “collapsed” configuration refers to the lowest profile configuration of the struts. In this context, “profile” refers to the distance away from the axis of the device that is spanned. Therefore, “low profile” refers to configurations in which the device is entirely within a small distance from the axis of the device. The “collapsed configuration” is the configuration in which the struts have the lowest possible profile, that is, where they lie as close as possible to the axis of the device. Having a low profile configuration simplifies insertion and removal of the device, and strut designs which tend to reduce the profile of the occlusion device are advantageous.

In the collapsed configuration, the embodiment shown in FIG. 1 would have the struts 28 and the occlusive member 26 positioned as close as possible to the longitudinal axis of the device, i.e. they would have the smallest possible cross-section. This configuration facilitates the deployment of the filter subassembly 14 by permitting easier delivery through the blood vessel 18 on the distal end of a catheter shaft, as well as easier retrieval of the filter subassembly 14 at the conclusion of the procedure. By minimizing the profile of the filter subassembly, this configuration is more easily passed through the vasculature leading to the filtration site from the insertion point.

When moved from the expanded configuration, shown in FIG. 1, into the collapsed configuration, the membrane 26 may not lie in the same profile as it did prior to deployment into the expanded configuration. This is because the membrane is retracted strictly by the action of the struts, and excess folds of material may extend from between the struts in the collapsed configuration. This may cause the profile of the filter subassembly 14 to be larger after retraction than it was prior to deployment. This enlarged profile can cause the membrane 26 to rub against the vessel walls in an undesirable manner. One way to address this difficulty is to use a retrieval catheter as described in Applicant's copending application entitled STRUT DESIGN FOR AN OCCLUSION DEVICE, application Ser. No. 09/505,546, filed Feb. 17, 2000, the entirety of which is hereby incorporated by reference.

In the “expanded” configuration shown in FIG. 1, the struts 28 and the occlusive member 26 are positioned such that they span substantially the entire width of the blood vessel 18 in which they are positioned. This is preferably the highest profile possible for the struts within the blood vessel. This configuration facilitates the use of the filter subassembly 14 to trap embolic matter while permitting passage of blood through the filter subassembly. By providing a means to span substantially the entire width of the blood vessel 18 to be filtered, the struts 28 support the occlusive member 26 in a configuration which forces the blood flow through the vessel to pass through the pores or openings in the filter subassembly 14 while retaining emboli therein. This produces the desired filtering effect.

Actuation of the struts in order to adjust the device from the collapsed configuration to the expanded configuration (shown in FIG. 1) is achieved using either a tension or a torsion mechanism. In tension based actuation, the pull wire 24 is displaced axially within the outer shaft member 22 in a proximal direction. In one preferred embodiment, this displacement allows the struts to expand under a built-in bias into the expanded configuration. In the embodiment shown in FIG. 1, the displacement applies an outward biasing force to the struts. In torsion based actuation, the pull wire 24 is rotated with respect to the outer shaft member 22, resulting in a rotational displacement which applies an outward biasing force to the struts. In order to adjust from the expanded to the collapsed configuration, the actuation is reversed, by either pushing or rotating the pull wire in the direction opposite from that used in the deployment, reversing the force upon the struts, and returning the device to the original configuration. Additional details are disclosed in the above-referenced STRUT DESIGN FOR AN OCCLUSION DEVICE.

G. Membrane

As seen in FIG. 1, the occlusive member or membrane 26 is preferably attached to each of the struts 28 and extends completely around the longitudinal axis of the device. Preferably, the occlusive member 26 is attached to the outer surface of the struts 28; however, it may be attached along the inside of the struts 28 as well. Moreover, it will be appreciated that the filter membrane may be provided inside some of the struts and outside of others. It will also be appreciated that struts may be provided on both sides of the membrane in a sandwiched configuration, or that two membranes may sandwich a set of struts.

At its distal end the occlusive member 26 is preferably joined to the strut hypotube 30, or, alternatively, to the guide tip 16. As the occlusive member 26 can be constructed in varying lengths, its proximal end may be located between the midpoint and the proximal end of the struts 28. Where the occlusive member 26 extends along the entire length of the struts 28 it may also be attached at its proximal end to the strut hypotube 30. Thus, when the struts 28 are radially expanded, the occlusive member 26 will likewise expand so as to take on a cross-sectional area corresponding approximately to that of the internal dimensions of the blood vessel 18. It is contemplated that the occlusive member can be joined to the struts 28 and strut hypotube 30 by employing standard attachment methods, such as heat fusing, adhesive bonding, etc.

One preferred occlusive member 26 is a nonelastomeric membrane with a number of pores which are approximately 20-100 microns in diameter. Suitable nonelastomeric materials include, but are not limited to: polyurethane, polyethylene, polyethylene terephthalate (PET), expanded polytetrafluoroethylene (PTFE), and polyether-based polyamides sold under the trade name PEBAX by Elf Atochem. This type of occlusive member may be extruded or dip molded, with the pores formed by the mold itself, or subsequently using an excimer laser or other drilling process.

One suitable elastomeric material is a block copolymer of styrene-ethylene-butylene-styrene (SEBS), available under the trade name C-FLEX, sold by Consolidated Polymer Technologies. The membrane may also be made from latex or silicone. The occlusive member may alternatively comprise a polymer mesh of polyurethane, nylon, polyester, or polyethylene, with pores approximately 30-50 microns in diameter. Yet another alternative is a braid of polyester or nitinol. To prevent formation of blood clots on the occlusive member, it may be coated with heparin or other known antithrombogenic agents such as hirudin or pirudin.

A variety of pore configurations are suitable for use with the occlusive member. First, where the membrane extends along the entire length of the struts, about 2-10 pores of about 20-200 microns diameter may be arranged longitudinally along the occlusive member to provide perfusion. Another suitable configuration for this type of occlusive member consists of several pores of about 20-200 microns in diameter on the distal half of the member, and large triangular, round, or square cutouts on the proximal half. Alternatively, the entire surface of the occlusive member may have pores of about 20-200 micron size. This configuration is also contemplated for use where the occlusive member 26 has an open proximal end. When using this type of occlusive member, a non-permeable cover or web may be placed over the juncture of the proximal ends of the struts to the distal shaft, to prevent formation of thrombi in the narrow passages formed at this point.

The membrane may be mounted on the device so as to create a loose or “baggy” portion of the membrane between proximal and distal points of attachment to the struts and to the strut hypotube/guide tip, respectively. In other words, the membrane may have a proximal point or region of attachment to the struts, a baggy portion distal of the proximal point of attachment in which the membrane is unattached to the device, and a distal point of attachment distal of the baggy portion. On such a membrane, the distal and proximal portions that are intended for attachment to the struts, guide tip and/or strut hypotube may preferably be substantially nonporous, to permit better adhesion. In one preferred embodiment, this membrane may have about 400 to 1000 pores, more preferably about 700-800 pores.

The membrane or occlusive member may also comprise a strut-deployable balloon that incorporates perfusion tubes which permit fluid communication (but not flow of emboli) between the proximal and distal sides of the balloon. The perfusion tubes may comprise lengths of tubing which terminate (at their proximal and distal ends, respectively) at points of intersection with the proximal and distal faces of the balloon. Alternatively, perfusion may be facilitated through the lumen of the outer shaft member via openings formed therein proximal of the balloon, and via the (porous) guide tip distal of the balloon. A valve system may be employed to regulate the flow of fluid through the lumen.

The device may also employ dual occlusive members on a single set of struts, with a proximal filter with relatively large pores and a distal filter with smaller pores. With any of the mentioned types of occlusive member, it is contemplated that an aspiration catheter may be employed to remove thrombi from the filter(s) at various points in an angioplasty or other similar procedure.

H. Guide Tip

As shown in FIG. 1, located most distally upon the shaft 12 is a guide tip 16. The guide tip lies distal of the filter subassembly 14 and provides a flexible leading extension which bends to follow the curvature of the blood vessels through which the device is advanced. By bending to follow the wall of the blood vessel, the guide tip 16 leads the filter subassembly 14 and other more proximal elements of the device in the direction of the tip so as to make the device move through the vessel without excessive impact against the walls of the blood vessels of the patient.

With further reference to FIG. 1, in one embodiment the guide tip 16 is formed by creating a rounded solder joint tip 35 to the pull wire 24 of the shaft 12, and wrapping it in a thinner wire to produce a coil which provides a spring force between the filter subassembly 14 and the rounded tip 35. The wire used for the coil 16 is preferably made of a radiopaque material. Because the pull wire 24 is constructed of a flexible material, such as nitinol, it will bend when the rounded tip 35 is pushed against the curving wall of a blood vessel. However, as the deflection of the tip increases, the spring force of the coil of thinner wire will urge the filter subassembly 14 and shaft 12 into alignment with the guide tip 16. In this way, the entire shaft is made to follow the path of the guide tip 16 as it advances through the blood vessels toward the treatment site.

I. Operation

The use of the described embodiments of the instant invention will generally be part of a process of therapy on a portion of the blood vessel of a patient. Usually, the therapy will involve treatment of some form of blockage of the blood vessel. However, those skilled in the art will recognize that the use of the described invention is appropriate in any situation where there is a possibility of embolic matter being dislodged from the vasculature of the patient, and therefore a desire to inhibit the dispersal of such embolic matter into the bloodstream of the patient.

As used herein, “method” refers to a preferred sequence used to accomplish a goal. Furthermore, the method which is described below is not limited to the exact sequence described. Other sequences of events or simultaneous performance of the described steps may be used when practicing the instant invention.

First, the device is manipulated so that the filter subassembly or subassemblies are in the collapsed position. This simplifies the insertion of the device into the blood stream of the patient. The device is then inserted through an insertion site into a blood vessel of the patient. Once inserted into the vasculature of the patient, the device is advanced distally until the distal portion of the device is located adjacent to the region of the blood vessel to be treated.

The device is positioned such that the filter subassembly lies generally downstream of the treatment site, or more generally, such that the filter subassembly lies between the treatment site and any site which is of particular susceptibility to embolic damage (e.g., the brain or coronary arteries). In this way, the filter is positioned so as to intercept any embolic matter dislodged at the treatment site, before such embolic material can reach any vulnerable area or be dispersed through the blood flow of the patient.

Once in position, the filter subassembly is actuated so that it assumes its expanded configuration, effectively occluding the blood vessel so that all blood flow must pass through at least one of the filter membranes or other occlusive members of the device.

The desired therapy is now performed upon the region of the blood vessel to be treated. This may involve placement or removal of support stents, balloon angioplasty, or any other vascular therapy that is conducted through the use of interventional techniques. In the course of such interventional treatment, additional catheters or other devices may be introduced to the treatment area by threading them over or along the shaft of the occlusive device. During the therapy, any embolic matter which is dislodged will flow into the filter and be caught by the membranes supported by the struts.

At any point during the therapy, the embolic matter may be aspirated from the filters through the use of separate aspiration catheters or through the lumen of the outer hypotubes forming the shaft of the occlusive device. Such aspiration may be repeated as often as necessary to maintain perfusive blood flow through the filter subassembly and treated region.

When the therapy is concluded, the filter subassembly is retracted into its collapsed configuration by reversing the actuation process. This will return the struts to a low profile which can then be withdrawn from the patient through the insertion site.

II. Back End Support

As previously described above, the struts 28 of the filter device shown in FIG. 1 are expanded and retracted as a function of the pull wire's 24 relative longitudinal motion to the hypotube 22. Due to the relative lack of rigidity of the pull wire 24, there is a greater susceptibility to kinking and/or bending of the pull wire 24 at the insertion point of the hypotube 22 upon manipulation of its relative position. Thus, it is preferable to provide some means of support at the proximal end of the distal hypotube 22 to account for possible damage to the pull wire 24 during operation.

In a preferred embodiment of a back-end support design 100 which would provide this means of support, shown in FIGS. 6 and 7 above, the hypotube 22 can be considered to be a distal hypotube. A coil 102 is bonded to the proximal end of the distal hypotube 22, and spans a certain distance over the pull wire 24 which extends proximally out of the distal hypotube. A proximal hypotube 104 is provided over the pull wire 24, to which the coil 102 is also bonded. The bonding of the coil 102 to the distal and proximal hypotubes can be done using a variety of methods, but one preferred method is by soldering (shown in the drawings by shaded hatch marks). On the distal end of the coil 102, solder is applied to the last couple spirals of the coil 102 to hold them in place and partially bond them to the distal hypotube 22. The solder is preferably applied carefully so as to not accidentally include the pull wire 24 in the bonding at this point. Alternatively, the coil 102 may be bonded to the distal hypotube prior to insertion of the pull wire 24.

The coil 102 is preferably in a compressed configuration when the struts are closed, i.e., when the struts lie closest to the axis of the distal hypotube 22 (FIG. 6). Thus, when the struts are expanded, the coil 102 is in a more relaxed state (FIG. 7). A polyimide sheath 201 is preferably bonded by adhesive over the soldered distal end of the coil 102 and the proximal end of the distal hypotube 22. The solder enclosure of the coil 102 to the hypotube 22 preferably provides a solid surface to which this protective tube 201 may be bonded. This polyimide sheath 201, which may be also be made of other polymers such as PET or thin-walled metals, enhances the connection that holds the coil 102 to the distal hypotube 22. On the proximal end of the coil 102, solder is preferably applied much more liberally, bonding the last few spirals of the coil 102 to both the proximal hypotube 104 as well as the pull wire 24. The proximal hypotube 104 is crimped in preferably at least two places, and as illustrated at four places both in the proximal and distal ends of the proximal hypotube 104, to the pull wire 24 proximally of the coil 102. This further enforces the connection between the proximal hypotube 104 and pull wire 24 and thus allows the pull wire 24 to be manipulated by manipulating the proximal hypotube 104.

One advantage of this embodiment is to provide external support to the pull wire 24 outside of the distal hypotube 22, to diminish the chance of kinking or bending when the pull wire is pushed into the hypotube. The coil also assists in preventing overexpansion of the struts, because when the proximal hypotube 104 and thus the pull wire 24 are pulled too far away relatively from the distal hypotube, the force of the coil 102 prevents this displacement from being too large.

Thus, when the proximal hypotube 104, and thus the pull wire 24, is moved in the proximal direction relative to the distal hypotube 22, it deploys the occlusion filter, as well as stretches the length of the coil 102 as shown in FIG. 7. When the filter subassembly is fully deployed to occlude a blood vessel, the coil 102 is preferably in its relaxed configuration. Therefore, any further stretching of the coil as the proximal hypotube 104 is moved proximally will cause the coil 102 to exert a force tending to bring the proximal and distal hypotubes closer together; this advantageously operates to prevent the filter to become overexpanded.

The materials and dimensions for one preferred embodiment are as follows. The coil 102 is preferably constructed of stainless steel, but platinum and nitinol may also be used. Its length may vary from roughly between about ¼ inch to 1 inch, and in one embodiment is about 14 mm when relaxed (i.e., as in FIG. 7). The coil in one embodiment has a diameter of about 0.014 inches, and an inner diameter of about 0.009 inches. Both the distal hypotube 22 and the proximal hypotube 104 in this embodiment have an outer diameter of about 0.014 inches, and an inner diameter of about 0.009 inches. The hypotubes are constructed preferably of nitinol, but other metals or materials may be used as well. Furthermore, the proximal hypotube and distal hypotube do not need to be made of the same material. Thus, in one embodiment the distal hypotube may be made of nitinol and the proximal hypotube may be made of stainless steel. In addition, the majority of the distal hypotube 22 is preferably protected by a PTFE coating. The polyimide sheath 201 has a preferred length of about 4 mm, with about 3 mm overlapping the proximal end of the distal hypotube 22. In addition to the preferred material of polyimide, the sheath 201 may be made from PET heat shrink tubing, or any other thin walled polymer or metal.

The proximal hypotube in one embodiment has a length of about 7.5 mm, with the most proximal crimp being located about 2 mm from the proximal end and the most distal crimp being located about 1.5 mm from the distal end. The coil 102 is preferably soldered over a length of about 2 mm of each end of the coil.

As a slight variation to the above embodiment, a second polyimide or other polymer sheath 202 or heat shrink tubing may be bonded to the proximal end of the coil 102 in addition to the distal end of the coil 102, for reinforcement purposes. This variation is shown in FIGS. 10 and 11, which illustrates the device in its closed and open configurations, respectively.

In another embodiment shown in FIGS. 12 and 13, the pull wire 24 tapers 216 to a wider outer diameter at the proximal end of the coil 102. In a preferred design of this embodiment, the outer diameter of the pull wire 24 tapers 216 from about 0.0086 to approximately 0.013 or 0.014 inches over a short distance at the area where the coil 102 is bonded, although these dimensions can be modified. The coil 102 is soldered to the pull wire 24 at this tapering point 206, and is also soldered to the distal hypotube 22. In this embodiment, the pull wire 24 is held directly by the operator instead of being crimped to and manipulated by a proximal hypotube 104 as in the earlier described embodiment. The pull wire 24 can be moved back and forth relative to the distal hypotube 22, with the coil 102 to protect it from kinking and to keep it within a specified range of motion. FIG. 12 shows this embodiment in its collapsed mode, while FIG. 13 is in its expanded mode.

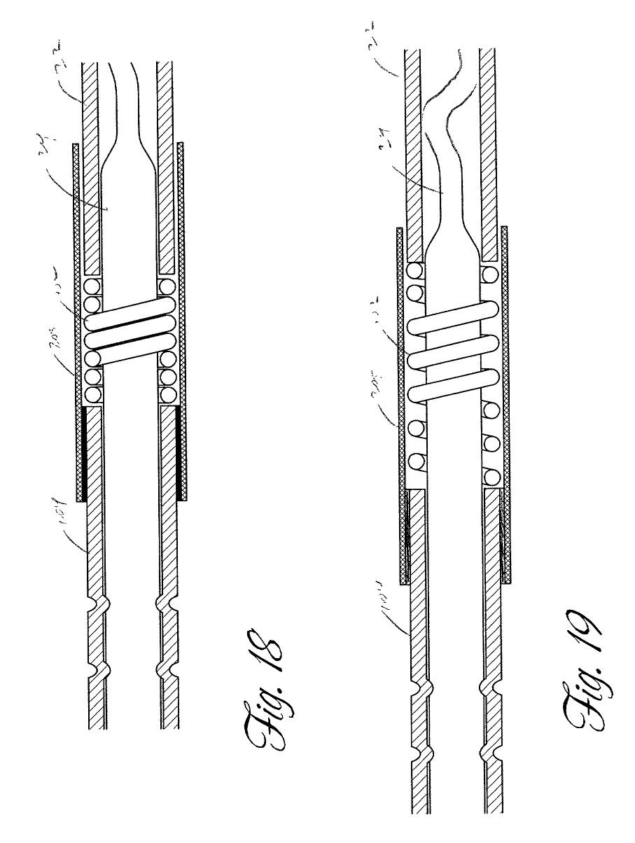

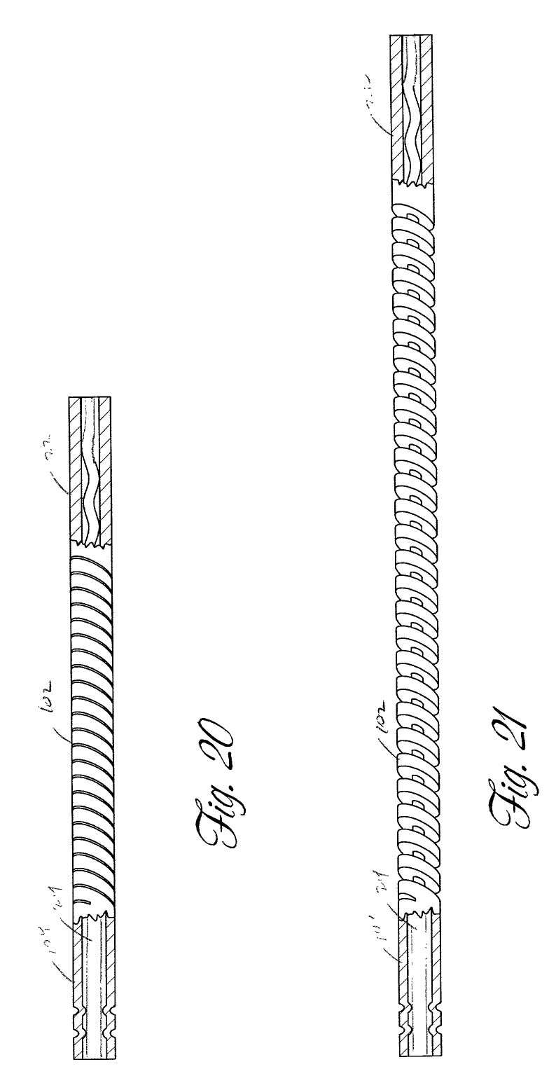

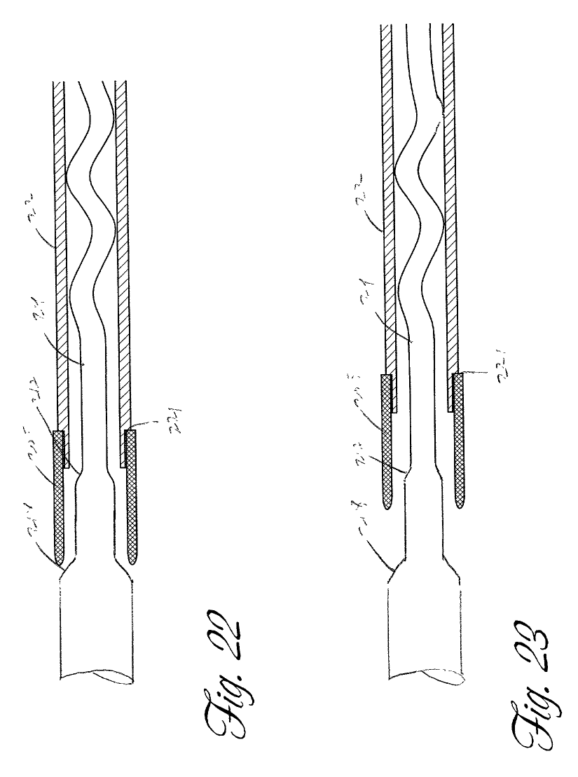

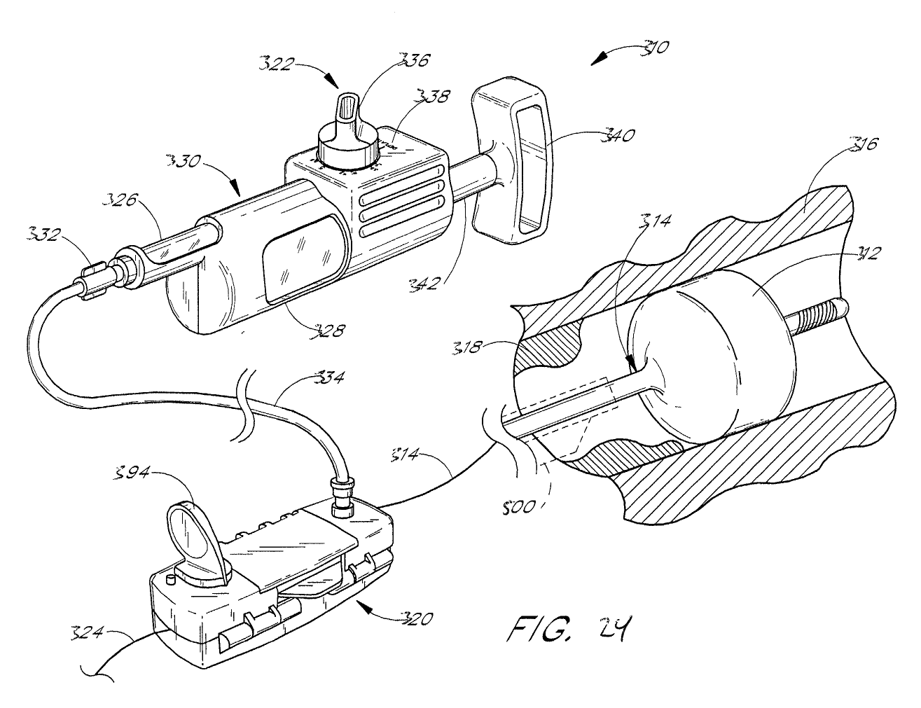

FIGS. 14 and 15 show a slight variation of the previous embodiment, with a protector tube 205 covering the majority of the coil 102 when the device is contracted. The first two or three spirals of the coil 102 on its distal end my still be soldered together, or the coil 102 may simply be bonded with adhesive to the distal hypotube 22. The protector tube 205 can then be bonded using adhesive or similar methods to the proximal end of the distal hypotube 22 and optionally to the distal end of the coil 102 as well. The function of the tube 205 is to provide an extra support to prevent the pull wire 24 from kinking upon manipulation relative to the distal hypotube 22. The tube 205 can be made of any thin-walled polymer or metal, as described above.

FIGS. 16 and 17 show yet another embodiment of a back-end support system, in which a coil 102 is not utilized. A proximal hypotube 104 is crimped to the pull wire 24 as in the other embodiments, and a protective tube or sheet support 205 is bonded with adhesive or by other means to the outside of the proximal hypotube 104. This sheet support may be made of nitinol, stainless steel or other suitable material, and may be attached by crimping to the proximal hypotube. As illustrated in FIG. 17, this protective tube spans across the gap between the two hypotubes when the device is in its expanded mode. FIG. 16 shows the collapsed mode, in which the gap between the two hypotubes is closed and the two hypotube abut against one another. As in FIGS. 14 and 15, this protector tube 205 functions as a wall support to prevent kinking and/or bending of the pull wire 24 upon manipulation relative to the proximal hypotube 104. The pull wire 24 in this embodiment is also preferably tapered, with a larger diameter at its proximal end to improve the column strength of the wire.

A variation to this embodiment is somewhat of a combination between the preferred embodiment of FIGS. 6 and 7 and the previous embodiment of FIGS. 16 and 17. This variation utilizes both the long protector tube 205 as well as the coil 102, and both may be affixed in a variety of methods as described above. This embodiment is shown in FIG. 18 in its collapsed mode, and FIG. 19 in its expanded mode.

In yet another embodiment of the present invention shown in FIGS. 20 and 21, a single hypotube is laser cut near its proximal end to form a natural coil 102 which serves to protect the pull wire 24. The hypotube is crimped to the pull wire 24 proximal to the natural coil 220 section in a proximal hypotube section 104, such that by manipulating the proximal section 104 of the hypotube relative to the distal end, the occlusion device can be opened and closed. This design advantageously eliminates the use of outside parts such as a metal coil, solder, or protective tubing. The pull wire 24 is manipulated by grasping the proximal end of the hypotube, and in moving it relative to the distal end of the hypotube, the natural coil 220 is expanded or contracted. FIG. 20 shows the device in its contracted mode, while FIG. 21 shows it in is expanded mode.

In yet another preferred embodiment shown in FIGS. 22 and 23, the pull wire 24 comprises two tapers 217 and 218 which both increase the diameter of the pull wire 24 towards its proximal end. Additionally a thin-walled protector tube 205 is bonded to and extends past the proximal end of the hypotube 22 to provide support against kinking and/or bending of the pull wire 24. The more distal taper 217 of the pull wire 24 expands the diameter to a transition diameter just larger than the inner diameter of the hypotube 22. The proximal taper 218 expands the diameter to be larger than the inner diameter of the protector tube 205, preferably the same as the outer diameter of the hypotube 22, preventing the pull wire 24 from being pushed into the hypotube 22 past its tapers. Preferably, this thin walled hypotube 205 is attached to the proximal end of the shaft 22 such that when the struts of the device are in a closed configuration, the thin walled hypotube extends over the length of the section of constant cross-section between the two tapers. The shaft 22 preferably has an outer wall ground with a recess 221 at its proximal end to accommodate placement of the thin walled hypotube without significantly increasing the profile of the device. The tapers 217 and 218 as well as the protector tube 205 together act to strengthen and support the pull wire 24 to prevent kinking. As illustrated, the protector tube 205 may be seated in a recess or indentation 221 in the proximal end of the hypotube 22.

FIG. 23 illustrates the filter device with the pull wire 24 moved to open the struts (not shown). Moving the pull wire 24 proximally away from the shaft 22 causes the section of constant cross-section between the two tapers to be outside the rigid thin walled hypotube. However, because of the protection to the pull wire provided by the thin walled hypotube and the added dimension due to the tapers at the proximal end of the pull wire, the tendency of the pull wire to kink or bend is significantly reduced. The tapers and support hypotube together act to strengthen and support the pull wire to prevent kicking and/or bending during operation of the occlusion device.

III. Occlusion Balloon System Utilizing Back End Support

It will be appreciated that the embodiments described above for providing kink protection to a pull wire that is inserted into a hypotube can be applied to other devices having a similar structure. For example, these embodiments can be applied to any device having a first elongate body slidable inside a second elongate body. As described below, one device that these embodiments may have particular applicability to is an occlusive device for use in an occlusion balloon system. In this system, a valve is inserted into a hollow guidewire to control inflation of a balloon on the guidewire. This valve comprises an elongate body which can be modified to include a coil, a proximal hypotube, tapers, or any of the features described for the pull wire above, in order to provide the valve with kink protection. Further details regarding the general features of this system will now be described.

A. Balloon System

FIG. 24 illustrates generally the components of one exemplifying occlusion balloon guidewire system 310. As described in further detail below, an occlusion balloon 312 used in this system is delivered on a guidewire 314 to a location in a blood vessel 316 distal an occlusion 318. Through the use of an adapter 320 and an inflation/deflation device or syringe assembly 322, the balloon is inflated through a lumen in the guidewire 314 to occlude the vessel distal to the occlusion. Through the use of a valve 324 described below, the adapter 320 can be removed from the proximal end of the guidewire 314 while the balloon 312 remains inflated. With the proximal end of the guidewire free of obstructions, various therapy and other catheters can be delivered and exchanged over the guidewire 314 to perform treatment on the occlusion 318. Because the balloon 312 on the guidewire 314 remains inflated distal to the occlusion 318, any particles broken off by treating the occlusion 318 are isolated proximal to the balloon. These particles can be removed using an aspiration catheter 500 (shown in phantom in FIG. 24) delivered over the guidewire. After the particles are removed, the adapter 320 and inflation/deflation device 322 can be reattached to the proximal end of the guidewire to deflate the balloon.

B. Syringe Assembly

Preferred embodiments of the present invention may comprise or be used in conjunction with a syringe assembly as described in U.S. Pat. No. 6,234,996, the entirety of which is incorporated herein by reference in its entirety.

One preferred embodiment of a syringe assembly 322 for inflation and deflation of an occlusion balloon is shown in FIG. 24. The syringe assembly 322 comprises a low-volume inflation syringe 326 and a high capacity or reservoir syringe 328 encased together in a housing 330. The syringe assembly 322 is preferably attached via a connector 332 and a short tube 334 to an adapter 320 within which a low profile catheter valve 324 and a balloon catheter 314 are engaged during use. The balloon catheter is shown in an inflated state within a blood vessel in FIG. 24. An inflation/deflation knob 336 is disposed on the outside of the housing 330. Indicia 338 are preferably located on the housing 330 adjacent the knob 336 so that a clinician using the device can monitor the precise volume of liquid delivered by the inflation syringe 322. As depicted, the indicia 338 preferably comprise numbers corresponding to the size and shape of the balloon used. When the knob 338 is rotated from the “DEFLATE” or “0:” position to the number corresponding to the balloon in use, the syringe assembly 322 delivers the fluid volume associated with that balloon size. Alternatively, the indicia 338 could indicate the standard or metric volume of fluid delivered at each position. A handle 340 is formed at a proximal end of the plunger 342. Preferably, the handle 340 is large, as illustrated in FIG. 24, and is easily held in a clinician's hand.

C. Occlusion Balloon Guidewire

The occlusion balloon guidewire system generally illustrated in FIG. 24 performs the function of occluding a vessel and allowing for the slidable insertion or advancement of various other catheters and devices. The term “catheter” as used herein is therefore intended to include both guidewires and catheters with these desired characteristics. The term “occlusion” refers to both partial and total occlusion of a vessel.

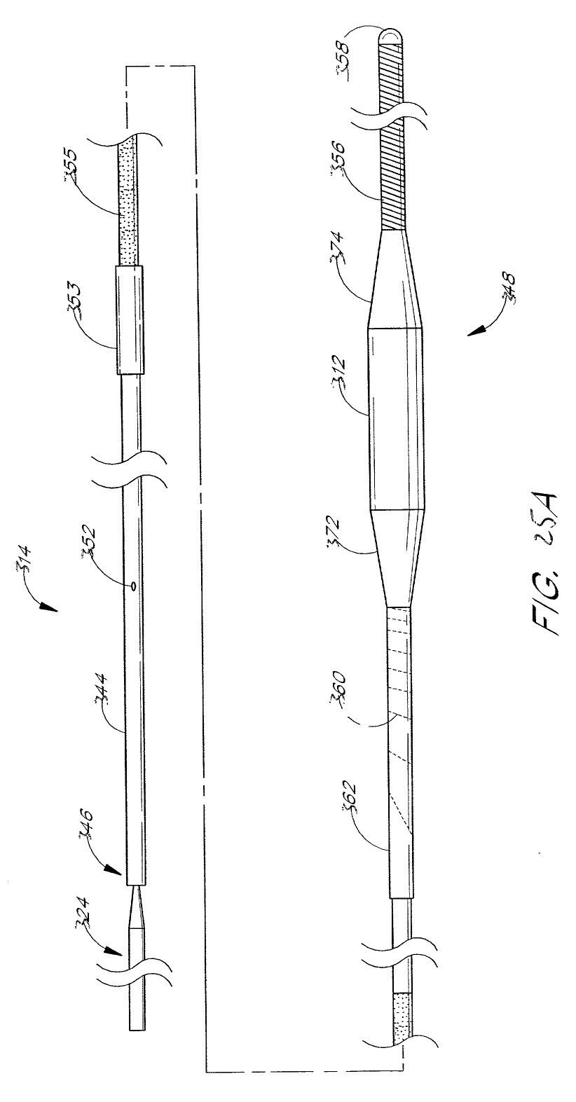

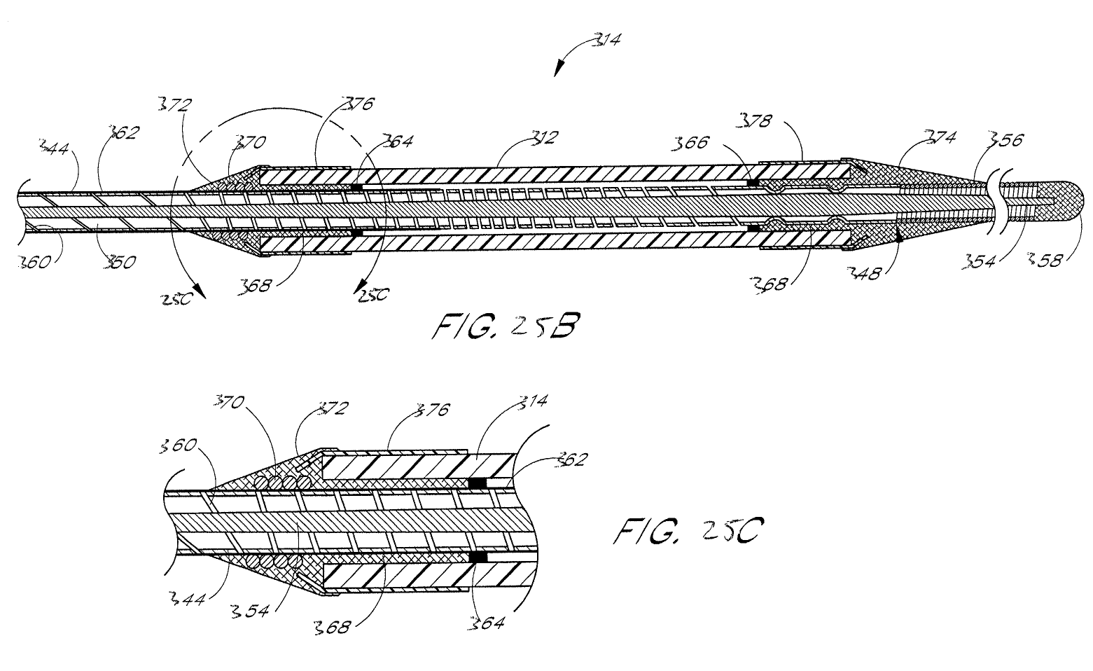

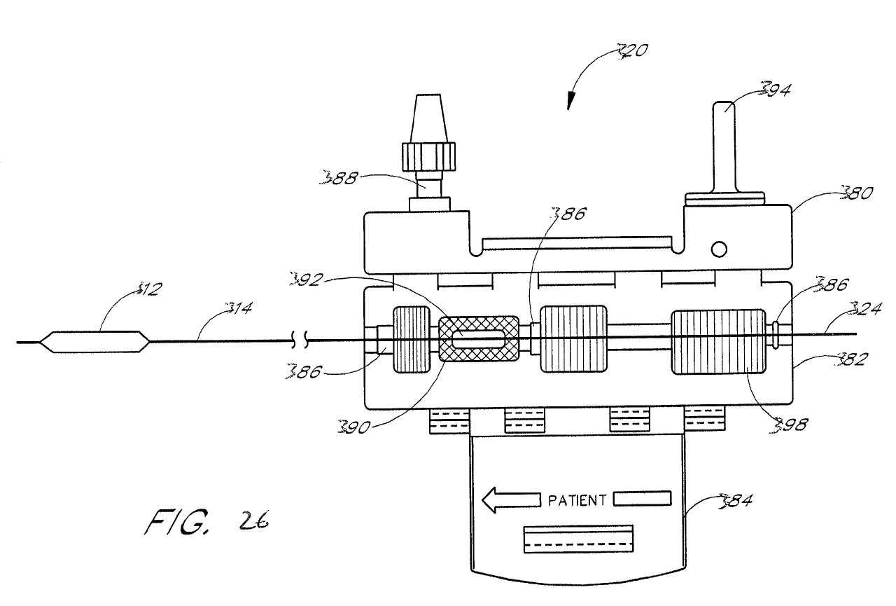

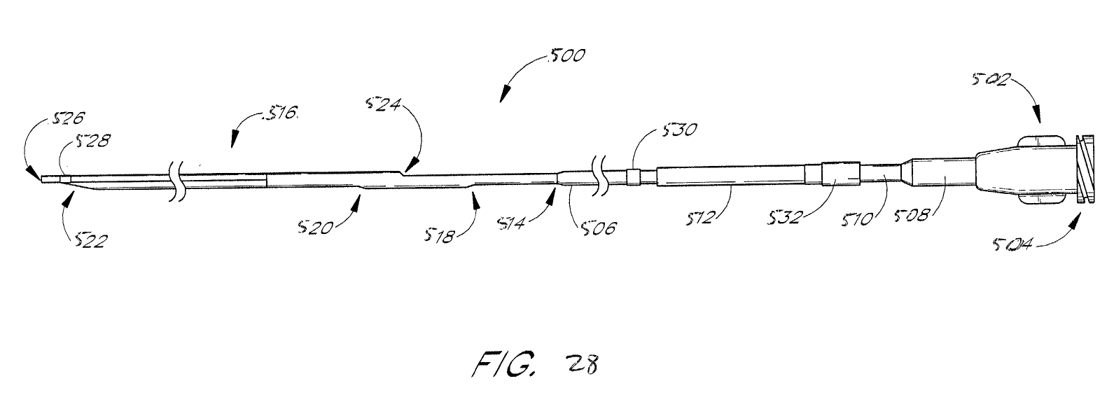

As shown in FIG. 25A, a balloon guidewire catheter 314 generally comprises an elongate flexible tubular body 344 extending between a proximal control end 346, corresponding to a proximal section of the tubular body 344, and a distal functional end 350 (not shown), corresponding to a distal section of tubular body 344. Tubular body 344 has a central lumen 348, which extends between the proximal and distal ends. An inflation port 352, shown also in FIGS. 27A and 27B described below, is provided on tubular body 344 near the proximal end 346. Inflation port 352 is in fluid communication with lumen 350 such that fluid passing through inflation port 352 into or out of the lumen 350 may be used to inflate or deflate an inflatable balloon 312 in communication with lumen 350.

A valve 324, as described below, is inserted into the proximal end 346 of the tubular body 344 to control inflation of a balloon 312 mounted on the distal end of the tubular body through inflation notch 352. The inflation notch 352 is preferably formed by electric discharge machining (EDM). A proximal marker 353, which is preferably made of gold, is placed over the tubular body 344 distal to the inflation notch 352. Distal to the marker 353, a nonuniform coating 355 of polymer material, more preferably polytetrafluoroethylene (TFE), is applied to the tubular body 344, terminating proximal to a shrink tubing 362. The shrink tubing 362 extends up to and within the balloon 312, as described below. Adhesive tapers 372 and 374 extend from the proximal and distal ends of the balloon, respectively. The proximal taper 372 preferably extends from the proximal end of the balloon to the shrink tubing 362 on the tubular body 344, while the distal taper 374 extends to coils 356 extending from the distal end 348 of the tubular body 344. The coils 352 terminate in a distal ball 358.

The length of the tubular body 344 may be varied considerably depending on the desired application. For example, when catheter 314 serves as a guidewire for other catheters in a conventional percutaneous transluminal coronary angioplasty procedure involving femoral artery access, tubular body 344 is comprised of a hollow hypotube having a length in the range from about 160 to about 320 centimeters, with a length of about 180 centimeters being optimal for a single operator device, or 300 centimeters for over the wire applications. Alternatively, for a different treatment procedure not requiring as long a length of tubular body 344, shorter lengths of tubular body 344 may be provided.

Tubular body 344 generally has a circular cross-sectional configuration with an outer diameter within the range from about 0.008 inches to 0.14 inches. In applications where catheter 314 is to be used as a guidewire for other catheters, the outer diameter of tubular body 344 ranges from 0.010 inches to 0.038 inches and preferably is about 0.014 to 0.020 inches in outer diameter or smaller. Noncircular cross-sectional configurations of lumen 350 can also be adapted for use with the catheter 314. For example, triangular, rectangular, oval and other noncircular cross-sectional configurations are also easily incorporated for use with the preferred embodiments, as will be appreciated by those of skill in the art. The tubular body 344 may also have variable cross-sections.

The tubular body 344 has sufficient structural integrity or “pushability” to permit catheter 314 to be advanced through the vasculature of a patient to distal arterial locations without buckling or undesirable kinking of tubular body 344. It is also desirable for the tubular body 344 to have the ability to transmit torque such as in those embodiments where it may be desirable to rotate tubular body after insertion into a patient. A variety of biocompatible materials known by those of skill in the art to possess these properties and to be suitable for catheter manufacture may be used to produce tubular body 344. For example, tubular body 344 may be made of a stainless steel material such as ELGILOY™ or may be made of polymeric material such as PEEK, nylon, polyimide, polyamide, polyethylene or combinations thereof. In one preferred embodiment, the desired properties of structural integrity and torque transmission are achieved by forming the tubular body 344 out of an alloy of titanium and nickel, commonly referred to as nitinol. In a more preferred embodiment, the nitinol alloy used to form the tubular body 380 is comprised of about 50.8% nickel and the balance titanium, which is sold under the trade mark TINEL™ by Memry Corporation. It has been found that a catheter tubular body having this composition of nickel and titanium exhibits an improved combination of flexibility and kink-resistance in comparison to other materials.

Other details regarding construction of balloon guidewire catheters may be found in assignee's U.S. Pat. Nos. 6,068,623, 6,228,072, and copending applications entitled FLEXIBLE CATHETER, application Ser. No. 09/253,591, filed Feb. 22, 1999, and FLEXIBLE CATHETER WITH BALLOON SEAL BANDS, application Ser. No. 09/653,217, filed Aug. 31, 2000, all of which are hereby incorporated by reference in their entirety.

As illustrated in FIG. 25A, an expandable member such as an inflatable balloon 312 is mounted on the distal end 348 of tubular body 344. In one preferred embodiment, the balloon 312 is a compliant balloon formed of a material comprising a block polymer of styrene-ethylene-butylene-styrene (SEBS), as disclosed in assignee's copending application entitled BALLOON CATHETER AND METHOD OF MANUFACTURE, application Ser. No. 09/026,225, filed on Feb. 19, 1998, and in U.S. Pat. No. 5,868,705, the entirety of both of which are hereby incorporated by reference. The balloon 312 may be secured to the tubular body 344 by any means known to those skilled in the art, such as adhesives or heat bonding. For example, for attachment of a SEBS balloon to a nitinol tube, a primer such as 7701 LOCTITE™ by Loctite Corporation is preferably used along with cyanoacrylate adhesive such as LOCTITE-4011.

The balloon 312 described in the preferred embodiments preferably has a length of about 5 to 9 mm and more preferably about 6 to 8 mm. Other expandable members are suitable for the catheter 344, such as those disclosed in assignee's copending applications entitled OCCLUSION OF A VESSEL, Ser. No. 09/026,106, filed Feb. 19, 1998, OCCLUSION OF A VESSEL, Ser. No. 09/374,741, filed Aug. 13, 1999, OCCLUSION OF A VESSEL AND ADAPTER THEREFOR, Ser. No. 09/509,911, filed Feb. 17, 2000, MEMBRANES FOR OCCLUSION DEVICE AND METHODS AND APPARATUS FOR REDUCING CLOGGING, Ser. No. 09/505,554, filed Feb. 17, 2000, and STRUT DESIGN FOR AN OCCLUSION DEVICE, Ser. No. 09/505,546, filed Feb. 17, 2000, the entirety of each of which is hereby incorporated by reference.

With reference to FIG. 25B, a core wire 354 is provided inside the lumen 350 and is crimped to the tubular body 344. Coils 356 extend from the distal end of the tubular body 344, surround the core wire 354, and terminate in a distal ball 358. In one embodiment, the core wire may have one or more tapers, and can extend proximally into tubular body 344. Other details regarding the core wire are discussed in assignee's copending application entitled CATHETER CORE WIRE, Ser. No. 09/253,971, filed Feb. 22, 1999, the entirety of which is hereby incorporated by reference.

In one embodiment, shown in FIG. 25B, the tubular body 344 preferably has cuts 360 to create a coiled configuration. A sleeve 362 is preferably provided over the tubular body 344. Adhesive stops 364 and 366 are provided about 1 to 2 mm from the ends of the balloon, to control the wicking length of the adhesive 368 into the balloon working area. Balloon inflation is provided through the cuts 360 in the tubular body 344. A marker 370 is mounted to the tubular body 366 proximal of the balloon 312. Adhesive tapers 372 and 374 are provided adjacent the balloon 312 to provide a transition region between the tubular body 344 and balloon 312 at the balloon's proximal end and between the balloon 312 and the core wire 354 at the balloon's distal end. Seal bands 376 and 378 are applied to the proximal and distal ends of the balloon to improve bond integrity. Other details regarding this balloon catheter may be found in assignee's above-referenced copending applications entitled FLEXIBLE CATHETER and FLEXIBLE CATHETER WITH BALLOON SEAL BANDS.

D. Inflation Adapter and Low Profile Catheter Valve

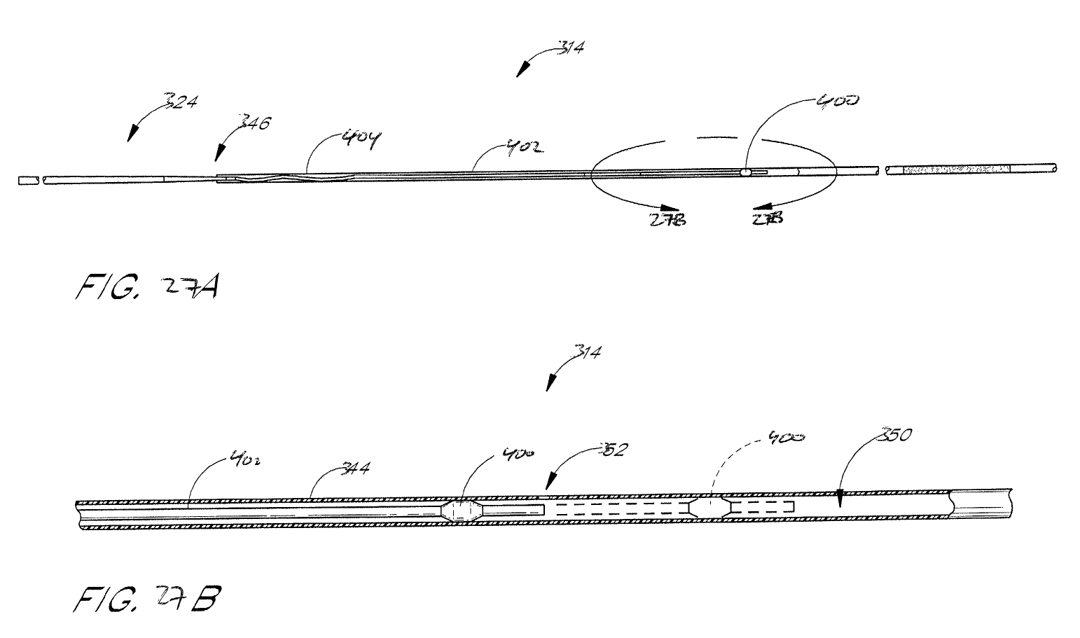

Referring next to FIG. 26, the inflation adapter 320 comprises a housing having two halves 380, 382 preferably formed of metal, medical grade polycarbonate, or the like. The halves 380, 382 are attached by hinges to be separated or joined in a clam shell manner. A locking clip 384 secures the halves while the adapter 320 is in use. Clips 382 within the housing accept and securely hold the catheter 314 in a correct position. The male luer member 388 or another suitable connector, extends from a top of the housing to provide an inflation passageway. Seals 390 are provided within the housing and around an internal segment 392 of the inflation pathway to conduct the pressurized fluid provided by the syringe assembly 322. An actuator 394, shown in FIG. 24 at the top of the adapter housing 396, controls a cam which operates sliding panels 398 (FIG. 26) contained in the housing.

As shown in FIG. 24, a low profile catheter valve 324 is attached to an open proximal end of the catheter 314. Inflation fluid is injected through the adapter 320 and valve 324 into a lumen of the hollow catheter 314, and into the balloon 312. The inflation adapter 320 is used to open and close the valve 324 to regulate the inflation of the balloon 312 mounted on the distal end of the catheter 314.

It will be emphasized that other types of adapters and/or valves can be employed with the inflation syringe and/or syringe assembly described herein, in order to achieve rapid and accurate inflation/deflation of medical balloons or other non-balloon medical devices. Therefore, although the preferred embodiments are illustrated in connection with a low volume occlusion balloon 312, other types of balloons and non-balloon devices can benefit from the advantages of the invention described herein.

As shown in FIGS. 27A and 27B, the low profile catheter valve 324 comprises a movable sealer portion 400 attached at a distal end of a wire segment 402 and positioned within the inflation lumen 350 of the guidewire catheter 314. The wire 402 may be secured to a spring just within a proximal opening of the catheter 314. It will be noted that various spring or biasing arrangements may be utilized, including a zig-zag wire 404 which is formed on or replaces the wire segment 402 and which provides biasing force to the sealer portion 400 due to frictional engagement with the walls of the lumen 350. The sealer portion 400 forms a fluid tight seal with the inflation lumen 350 by firmly contacting the entire circumference of a section of the inflation lumen 350. The sealer portion 400 may be positioned proximally of the side-access inflation port 352 on the catheter as shown in FIG. 27B, to establish an unrestricted fluid pathway between the inflation port 352 and the inflatable balloon on the distal end. As desired, the clinician may move the sealer portion 400 to a position at or distal of the inflation port 352, as shown in phantom in FIG. 27B, thereby preventing any fluid from being introduced into or withdrawn from the lumen 350 via the inflation port 352. The valve 324 is considered “low profile” because it is no larger in cross-sectional diameter than the catheter 314 itself.

The valve 324 shown in FIG. 27A and 27B can be modified to include the structure of any of the kink protection systems of FIGS. 6-7 or 10-23 above. Thus, in these previously described figures, the inner wire which served as the pull wire for a filter device is now considered the wire segment 402 of the valve 324, with the sealer portion 400 (not shown) being provided distal the bends of the wire (e.g., reference number 38 in FIGS. 6 and 7 and reference number 404 in FIG. 27A. Using FIGS. 6 and 7 as an example, when the wire 24 is retracted proximally, as shown in FIG. 7, the sealer portion 400 is proximal to the side access inflation port 352, and the balloon can be inflated. When the wire is advanced distally, as shown in FIG. 6, the sealer portion 400 is distal to the inflation port 352 to seal the lumen 350. It will be appreciated that although the expanded coil 102 of FIG. 7 has been described herein as the relaxed configuration, the coil can also be biased such that the contracted configuration of FIG. 6 is the relaxed configuration.

Preferably, the catheter 314 is positioned within the housing of the adapter 320 with the valve closed, such that the side inflation port 352 is located in the sealed inflation area 392 of the housing. The catheter 314 is then positioned in the second half 382 of the adapter 320. A distal portion of the catheter 314 extends out of the housing and into the patient, and a proximal portion of the catheter including the catheter valve 324 extends out of the other side of the adapter 320. The adapter is closed, the locking clip 384 is secured, and a syringe assembly is attached. The actuator 394 is moved from a first position to a second position, such that the sliding panels 398 within the housing cause the valve 324 to be in an open position to allow fluid flow through the inflation port 352. A syringe assembly 322 is then used to inflate the balloon 312. Closing the valve 324 is accomplished by moving the actuator 396 from the second position back to the first position, such that the balloon inflation is maintained. Once the valve is closed the adapter may be removed and treatment and other catheters may be delivered over the guidewire.

Other inflation adapter/inflation syringe assemblies may also be used. Also, the adapter 320 can have additional features, such as a safety lock provided on the actuator knob 394 to prevent accidental opening when the adapter is being used and the catheter valve is open. In addition, the adapter can be provided with an overdrive system to overdrive a sealing member into a catheter. Details of these features and other inflation assemblies may be found in assignee's U.S. Pat. No. 6,050,972 and copending applications, SYRINGE AND METHOD FOR INFLATING LOW PROFILE CATHETER BALLOONS, application Ser. No. 09/025,991, filed Feb. 19, 1998, and LOW VOLUME SYRINGE AND METHOD FOR INFLATING SURGICAL BALLOONS, application Ser. No. 09/195,796, filed Nov. 19, 1998, all of which are incorporated by reference in their entirety.

E. Aspiration Catheter

The occlusion system described above advantageously enables an exchange of catheters over a guidewire while an occlusive device isolates particles within the blood vessel. For example, a therapy catheter can be delivered over the guidewire to perform treatment, and then be exchanged with an aspiration catheter to remove particles from the vessel. Further details of this exchange are described in assignee's copending application entitled EXCHANGE METHOD FOR EMBOLI CONTAINMENT, Ser. No. 09/049,712, filed Mar. 27, 1998, the entirety of which is hereby incorporated by reference.

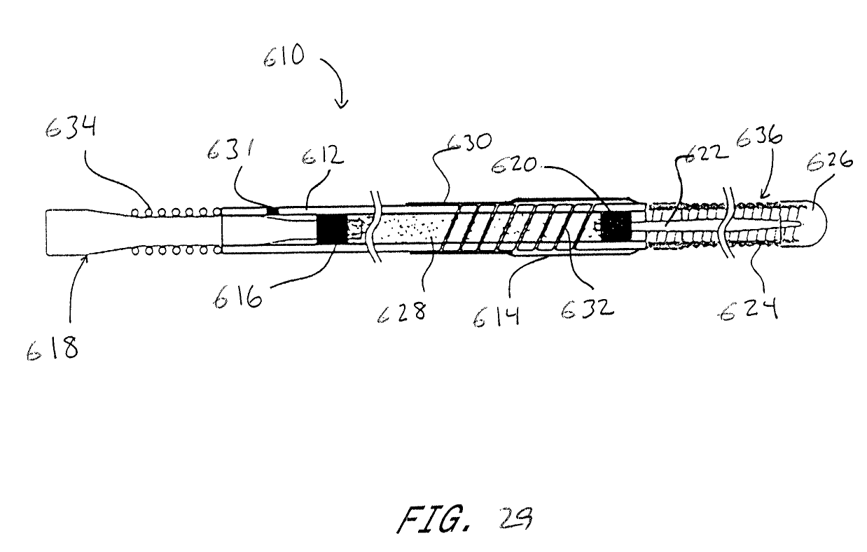

An aspiration catheter according to one preferred embodiment of the present invention is shown in FIG. 28. The catheter 500 includes an adapter 502 and an aspiration port 504 at its proximal end to which a source of negative pressure is attached. The aspiration catheter further comprises an elongate tubular body 506 which extends distally from the adapter 502 and through a plurality of support sheaths 510 and 512. Beyond the support sheath 512 the elongate tubular body 506 extends to a transition point 514 where the outer diameter of the tubular body 506 tapers down in size. This tapered or necked-down portion of the tubular body 506 is preferably inserted into a dual lumen tubing 516 through the proximal end 518 of the dual lumen tubing. The tubular body 506 is preferably inserted into one of the lumens of the dual lumen tubing 516 such that its distal end 520 is a sufficient distance distal from the proximal end 518 of the dual lumen tubing to provide a secure connection therebetween.

The dual lumen tubing 516 preferably defines two lumens, one for aspiration and the other for a guidewire to pass therethrough. More particularly, the lumen that the elongate body 506 is inserted into acts as the aspiration lumen, being in fluid communication with the lumen of the elongate tubular body 506. The aspiration lumen preferably ends in a distal aspiration mouth 522, which preferably defines an oblique opening. Aspiration therefore occurs through both the lumen of the elongate tubular body 506 and the aspiration lumen of the dual lumen tubing.

The guidewire lumen is provided adjacent the aspiration lumen in the dual lumen tubing and has a proximal end 524 preferably distal to the proximal end 518 of the aspiration lumen of the dual lumen tubing, and a distal end 526 preferably distal to the aspiration mouth 522. A marker 528 is placed within the guidewire lumen at the distal end of the aspiration mouth. Additional markers 530, 532 may also be placed over the elongate body 506 and/or support sheaths. Further details regarding these and other aspiration catheters are provided below and in Applicant's copending applications entitled ASPIRATION CATHETER, Ser. No. 09/454,522, filed Dec. 7, 1999, and U.S. Pat. No. 6,152,909, the entirety of both of which are hereby incorporated by reference.

IV. Self-Inflating Balloon System Utilizing Back End Support

FIG. 29 illustrated another embodiment of a balloon catheter utilizing a back end support such as described above. Although the embodiment shown herein describes a tapered pull wire with a coil provided thereover, it will be appreciated that the other embodiments described above for providing kink protection can also be used.

The balloon catheter 610 of FIG. 29 generally comprises an elongated tubular catheter body 612, an inflatable balloon 614 mounted along the distal end portion of the catheter body and an adjustable plunger 616 located in the lumen of the catheter body 612 at the proximal end. A pull wire or push wire 618 is coupled to the plunger 616 and provides a means for advancing and retracting the plunger 616 relative to the catheter body 612. The catheter body 612 is sealed fluid tight at the distal end by a marker/plug 620. The marker/plug 620 has a fixed position within the catheter body 612 and is preferably made of a radiopaque material to facilitate viewing of the balloon catheter during use.

A core wire 622 is attached to the marker/plug 620 and extends distally therefrom. A distal coil 624 extends around the core wire 622 to provide the balloon catheter with a flexible guide tip 636. The flexible guide tip 636 is adapted to facilitate the advancement of the balloon catheter 610 through the vasculature of the patient. A rounded cap 626 is provided over the distal end of the guide tip 636 such that the device will not damage the patient's tissue during advancement through the patient's vasculature.

The inflatable balloon 614 is mounted along the distal end of the catheter body 612, just proximal of the flexible guide tip 636. The distal end of the inflatable balloon 614 is attached directly to the catheter body 612. The proximal end of the inflatable balloon is attached to a length of shrink tubing 630. The shrink tubing 630 provides a surface on which the proximal end of the balloon can be securely mounted while maintaining a fluid tight seal therebetween.

The catheter body 612 includes a lumen in fluid communication with the inflatable balloon 616. Fluid exits the catheter body 612 and enters the balloon 616 through an opening in the catheter body 612 provided by a spiral slit 632 formed along the distal end portion. The spiral slit 632 causes the catheter body 612 to have a helical-shaped distal end portion. This helical-shape improves the flexibility of the catheter. In alternative embodiments, the catheter body may be provided with one or more exit ports along the distal end portion for allowing fluid to enter the balloon, rather than the spiral slit.

The adjustable plunger 616 is located within the lumen of the catheter body 612 at the proximal end. The plunger 616 is adapted to be advanced and retracted within the lumen for inflating and deflating the balloon 614. The interior region of the catheter body 612 between the plunger 616 and the marker/plug 620 defines a liquid trap 628 that may be filled with a fluid, such as a saline solution. A side-access port 631 is provided on the side of the catheter body 612 at a point distal to the proximal end. The side-access port 631 is in fluid communication with the lumen. Before, use, fluid is introduced into the liquid trap 628 through the side-access port 631 by retracting the plunger to a location proximal to the side-access port.

The wire 618 has a distal end portion that is coupled to the plunger 616. In the illustrated embodiment, the plunger 616 is constructed as an annular member defining an orifice at the center through which the wire 618 extends. It is preferred that the plunger 616 forms a fluid tight seal in the space between the outer diameter of the wire 618 and the inner diameter of the catheter body 612. Therefore, the fluid in the liquid trap 628 is prevented from escaping from the proximal end of the catheter body 612. The fluid tight seal is achieved by providing a plunger 616 that firmly contacts the inner circumference of the catheter body 612 along a substantial length of the plunger. The fit between the outer surface of the plunger 616 and the inner diameter of the catheter body 612 is tight. Moreover, the fluid tight seal between the plunger 616 and the catheter body 612 is preferably maintained at all times as the plunger is moved axially within the catheter body 612. However, the fit between the plunger 616 and catheter body 612 is preferably not be so tight as to prevent movement of the plunger 616 through the catheter body upon application of a sufficient longitudinal force on the wire 618.

The plunger 616 is preferably also capable of maintaining a seal at fluid pressures conventionally used to inflate catheter balloons, and should be capable of maintaining a seal at pressures that exceed conventional inflation pressures. Preferably, the plunger is capable of maintaining a seal at pressures up to about 10 atmospheres, and more preferably up to about 30 atmospheres, and most preferably at pressures up to about 60 atmospheres. The plunger is also preferably capable of undergoing multiple advancement and retraction cycles without losing the structural integrity required for maintaining a seal capable of withstanding pressures of from about 10 atmospheres to about 60 atmospheres. Optimally, the plunger is capable of undergoing at least 10, and preferably at least 20, advancement and retraction cycles while still being capable of maintaining a fluid tight seal at a pressure of 10 atmospheres.

In one preferred embodiment, the plunger 616 has an outside diameter slightly larger that the inner diameter of the lumen, but smaller than the outer diameter of the tubular body. With this configuration, the plunger may be tightly fit within the lumen through the proximal opening, to form a fluid tight seal at the proximal end of the catheter body 612.

The wire 618 preferably comprises a main shaft, an intermediate region, and a thin wire region coupled to the plunger 616. The intermediate region is preferably formed with a diameter slightly smaller than the inner diameter of the lumen in the catheter body. The main shaft has a larger diameter than the other portions to provide for increased bending stiffness and strength under tension and compression. The increased strength enables the technician to apply greater longitudinal forces to the wire 618, and therefore the plunger 616, without bending or breaking the main shaft of the wire 618.

During distal advancement, the wire 618 encounters compressive longitudinal forces caused by friction between the plunger and catheter body 612 and by the force of the fluid pressure acting on the plunger. A kink protection system prevents the intermediate region of the wire 618 from bending while longitudinal forces are applied to the wire. The kink protection system comprises a pre-expanded, wound helical coil 634 that surrounds the intermediate portion of the wire. The diameter of the kink protection coil 634 is smaller than the diameter of the main shaft of the wire, and is also smaller than the diameter of the catheter body 612. Therefore, as the wire 618 is advanced distally, the helical coil 634 is compressed between the main shaft and the proximal end of the catheter body 612. When the helical coil 634 becomes fully compressed, the main shaft is prevented from advancing further. In addition to protecting against kinking of the wire, this feature also advantageously provides a built-in stop mechanism that prevents the technician from advancing the plunger too far and thereby over-inflating the balloon. Accordingly, this safety feature eliminates the possibility of bursting the balloon within a blood vessel. This feature also reduces the possibility of damaging the blood vessel due to over-inflation.

Further details of this construction are described in Applicant's copending provisional application entitled METHOD AND APPARATUS FOR INFLATING SMALL BALLOONS, Ser. No. ______, filed on the same date as the present application, the entirety of which is hereby incorporated by reference.

Those skilled in the art will recognize that the described embodiments are given as examples only, and that numerous variations which fall within the scope of the present invention may be appropriate in order to use the invention depending upon the circumstances under which it is used. The description of preferred embodiments above should not be deemed to limit the scope of the present invention.

What is claimed is:

1. A medical device, comprising:
an elongate tubular body having a proximal end and a distal end and a lumen extending therethrough;
an inner wire within the lumen of the elongate tubular body having a proximal end extending proximal to the proximal end of the elongate tubular body and a distal end, the inner wire being moveable relative to the elongate tubular body;
an expandable member connected to the distal end of the elongate tubular body; and
a coil extending over the inner wire at a proximal end thereof, the coil being attached to the proximal end of the elongate tubular body.

2. The medical device of claim 1, further comprising a proximal hypotube connected to a proximal end of the coil, wherein the inner wire extends through the proximal hypotube.

3. The medical device of claim 2, further comprising a protective tubing over the proximal end of the coil and the distal end of the proximal hypotube.

4. The medical device of claim 1, wherein the proximal hypotube is crimped to the inner wire.

5. The medical device of claim 1, further comprising a protective tubing applied over the proximal end of the hypotube and distal end of the coil.

6. The medical device of claim 1, wherein the inner wire has a diameter proximal to the tubular body that is greater than the diameter of the lumen.

7. The medical device of claim 1, wherein the expandable member is an occlusive device having a proximal end connected to the tubular body and a distal end connected to the inner wire, wherein relative movement of the inner wire with respect to the tubular body causes the occlusive device to move from a nonexpanded configuration to an expanded configuration.

8. The medical device of claim 7, wherein the coil is compressed when the occlusive device is in a nonexpanded configuration.

9. The medical device of claim 1, wherein the expandable member is a balloon.

10. The medical device of claim 9, wherein the inner wire includes a valve for sealing the lumen of the elongate tubular body.

11. The medical device of claim 9, wherein the inner wire includes a plunger, and wherein advancing and retracting the plunger selectively inflates and deflates the balloon.

12. A medical device, comprising:
an elongate tubular body having a proximal end and a distal end and a lumen extending therethrough;
a pull wire within the lumen of the tubular body having a proximal end extending proximal to the proximal end of the tubular body and a distal end;
an expandable occlusive device having a proximal end connected to the tubular body and a distal end connected to the pull wire, wherein relative movement of the pull wire with respect to the tubular body causes the occlusive device to move from a nonexpanded configuration to an expanded configuration;
wherein the pull wire at its proximal end has at least one taper that increases the diameter of the pull wire to a size larger than the diameter of the lumen.

13. The medical device of claim 12, comprising two tapers at the proximal end of the pull wire, each of the tapers increasing the diameter of the pull wire proximally and separated by a section of constant diameter.

14. The medical device of claim 13, wherein when the occlusive device is in its nonexpanded configuration the more distal of the two tapers abuts against the proximal end of the tubular body.

15. The medical device of claim 14, further comprising a thin walled hypotube extending from the proximal end of the tubular body and extending over the section of constant diameter.

16. The medical device of claim 15, wherein the thin walled hypotube has an outer diameter that is substantially the same as that of the tubular body.

17. A medical device, comprising:
an elongate tubular body having a proximal end and a distal end and a lumen extending therethrough;
an inner wire within the lumen of the elongate tubular body having a proximal end extending proximal to the proximal end of the elongate tubular body and a distal end, the inner wire being moveable relative to the elongate tubular body;
an expandable member connected to the distal end of the elongate tubular body; and
a proximal hypotube attached to the proximal end of the inner wire.

18. The medical device of claim 17, wherein the expandable member is an occlusive device having a proximal end connected to the elongate tubular body and a distal end connected to the inner wire, wherein relative movement of the inner wire with respect to the tubular body causes the occlusive device to move from a nonexpanded configuration to an expanded configuration.

19. The medical device of claim 18, wherein when the occlusive device is in its expanded configuration, a gap is defined between the tubular body and the proximal hypotube.

20. The medical device of claim 17, further comprising a protective tubing extending over the gap and attached to the proximal hypotube.

21. The medical device of claim 17, further comprising a coil between the proximal hypotube and the tubular body.

22. The medical device of claim 21, wherein the proximal hypotube, the coil and the tubular body are integrally formed.

23. The medical device of claim 17, wherein the inner wire has a diameter at its proximal end that is larger than its diameter at its distal end.

24. The medical device of claim 17, wherein the expandable member is a balloon.

25. The medical device of claim 24, wherein the inner wire includes a valve for sealing the lumen of the elongate tubular body.

26. The medical device of claim 24, wherein the inner wire includes a plunger, and wherein advancing and retracting the plunger selectively inflates and deflates the balloon.
